(19) United States
(12) Patent Application Publication
(10) Pub. No.: US 2014/0024576 A1
(43) Pub. Date: Jan. 23, 2014

(54) HEPTOSE DERIVATIVES FOR USE IN THE TREATMENT OF BACTERIAL INFECTIONS

(71) Applicants: Vincent Gerusz, Paris (FR); Stéphane Vincent, Hyon (BE); Mayalen Oxoby, Braine-L'alleud (BE); Dmytro Atamanyuk, Chelles (FR); François Moreau, Orsay (FR); Mounir Andaloussi, Nieuwegein (NL); Abdellatif Tikad, Namur (BE)

(72) Inventors: Vincent Gerusz, Paris (FR); Stéphane Vincent, Hyon (BE); Mayalen Oxoby, Braine-L'alleud (BE); Dmytro Atamanyuk, Chelles (FR); François Moreau, Orsay (FR); Mounir Andaloussi, Nieuwegein (NL); Abdellatif Tikad, Namur (BE)

(73) Assignee: LABORATOIRE BIODIM, Paris (FR)

(21) Appl. No.: 13/990,728

(22) PCT Filed: Dec. 1, 2011
(86) PCT No.: PCT/IB2011/055404

§ 371 Date: Oct. 1, 2013

Related U.S. Application Data
(60) Provisional application No. 61/418,491, filed on Dec. 1, 2010.

(51) Int. Cl. C07H 3/02; A61K 45/06; A61K 31/7004

(52) U.S. Cl. CPC C07H 3/02; A61K 31/7004; A61K 45/06
USPC 514/2.4; 514/3.7; 514/23; 536/1.11

(57) ABSTRACT

Compounds having the general formula (I) pharmaceutical compositions containing them for use in inhibiting bacterial heptose biosynthesis and thereby lowering or suppressing bacterial virulence.

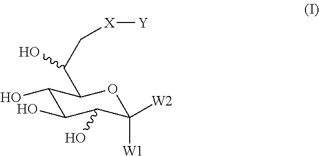

### CROSS-REFERENCE TO RELATED APPLICATIONS

This application is National Phase Entry of International Application No. PCT/IB2011/055404, filed on Dec. 1, 2011, which claims priority to U.S. Provisional Patent Application Ser. No. 61/418,491, filed on Dec. 1, 2010, both of which are incorporated by reference herein.

### BACKGROUND AND SUMMARY

The invention relates to new heptose derivatives, their preparation and intermediates, their use as drugs and pharmaceutical compositions containing them. The invention also relates to new heptose derivatives capable of inhibiting bacterial heptose biosynthesis and thereby lowering or suppressing bacterial virulence; as well as their antibacterial pharmaceutical applications. The invention particularly relates to new heptose derivatives capable of inhibiting the GmhA and/or HldE enzymes of bacterial heptose synthesis, thereby lowering or suppressing bacterial virulence; as well as their antibacterial pharmaceutical applications.

The lipopolysaccharide (LPS) is a major component of the outer membrane of Gram-negative bacteria. It is composed of three regions: the lipid A, the core oligosaccharide and the O antigen. The core oligosaccharide is divided into the inner core and the outer core. The inner core consists in a motif of five sugars: two Kdo (Kdo: 3-deoxy-D-manno-octulosonic acid) and three successive heptoses. The first heptose transfer is catalysed by the Heptosyltransferase I (protein WaaC) and the second heptose transfer by the Heptosyltransferase II (protein WaaF). The natural donor substrate of these transferases is ADP heptose, which is synthesized in bacteria from sedoheptulose-7-phosphate by the successive enzymatic steps catalyzed by the following enzymes: GmhA, HldE-K (former or other nomenclature: RfaE-K), GmhB, HldE-AT (former or other nomenclature: RfaE-AT) and HldD (former or other nomenclature: RfaD, WaaD) (Journal of Bacteriology, 2002, 184, 363).

Heptose synthetic pathway is conserved among Gram negative bacterial species and is necessary for full LPS synthesis. It has been demonstrated that a complete LPS is necessary for Gram negative bacterial pathogenesis. Bacteria lacking heptoses display a so-called “deep-rough phenotype” due to the absence of the O-antigen. While still able to survive as the commensal flora, they are unable to give a productive infection in the host and are very sensitive to detergents or hydrophobic antibiotics as well as to the bactericidal effect of the host complement (Annu. Rev. Biochem. 2002, 635).

Inhibitors of bacterial heptose synthesis are expected to prevent full LPS development in Gram negative bacteria, inducing a high sensitivity to the host complement and preventing or inhibiting bacterial infection. Small molecules inhibitors of heptose synthesis may therefore provide a novel way to treat bloodstream infections caused by pathogenic Gram negative bacteria, without affecting the commensal flora and with less selective pressure than conventional antibacterial agents.

A few inhibitors of bacterial heptose synthesis have been reported in the literature, especially on HldE (Chem. Biol. 2006, 437; Bioorg. Med. Chem. 2009, 1276; WO2008038136; WO2010001220) and Waac/Waaf (Bioorg. Med. Chem. Lett. 2008, 4022; Chem. Eur. J. 2008, 9530; WO2006058796). However, despite their attractiveness, these bacterial targets are still largely unexploited at this time since there are no drugs on market or on advanced clinical phases. One of the purposes of the present invention is therefore to provide novel compounds active on these targets.

The invention relates to new compounds having the general formula (I)

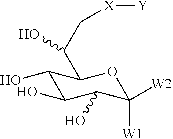

wherein,
Carbon-2 may be in D-manno-heptose or D-gluco-heptose configuration or as a mixture of both;
Carbon-6 may be in L-glycero-heptose or D-glycero-heptose configuration or as a mixture of both;
X is O, S, CH₂, CHF, CF₂ or NH;
Y is H or P(O)(OZ1)(OZ2), P(O)(OZ1)(NHZ2) or SO₂(OZ1);
Z1 and Z2, identical or different, are H, (C₁-C₆)alkyl, n-octadecanoyl, (C₁-C₆)fluoroalkyl, CH₂O(CO)O(C₁-C₆)alkyl, CH((C₁-C₆)alkyl)O(CO)O(C₁-C₆)alkyl, CH₂O(CO)O(C₁-C₆)fluoroalkyl, CH₂O(CO)(C₁-C₆)alkyl, CH((C₁-C₆)alkyl)O(CO)(C₁-C₆)alkyl, CH₂O(CO)(C₁-C₆)fluoroalkyl, CH₂CH(O-n-decanoyl)CH₂S-n-dodecanoyl, (C₂-C₆) alkenyl, (C₂-C₆) alkynyl, CH₂CH₂S(CO)(C₁-C₆)alkyl, CH((C₁-C₆)alkyl)(CO)O(C₁-C₆)alkyl, CH₂(CO)O(C₁-C₆)alkyl; phenyl optionally substituted by one or several identical or different groups R; 4-6 membered monocyclic saturated or unsaturated heterocycle containing 1-3 heteroatoms selected from N, O and S, optionally substituted by one or several identical or different groups R; or mono-, di- or trivalent cation such as lithium, sodium, potassium, magnesium, calcium, cesium, barium, ammonium, to form a phosphate salt; Z1 and Z2 may form a 4-10 membered cycle with each other, optionally including those selected from the group comprising CH₂CH₂CH(m-chlorophenyl or pyridyl), CH₂CH₂CH(O(CO)(C₁-C₆)alkyl);

W1 and W2 identical or different, optionally linked with each other, are selected from the group consisting of H, F, CN, (C₁-C₆)alkyl, (C₁-C₆)alkyl-ORₐ, (C₁-C₆)alkyl-O(C₁-C₆)alkyl, (C₁-C₆)fluoroalkyl, (C₂-C₆)alkenyl, (C₂-C₆)alkynyl, phenyl, 4-10 membered monocyclic or bicyclic saturated or unsaturated heterocycle containing 1-5 heteroatoms selected from N, O and S; CO₂Rₐ, CORₐ, CONRₐR_{b}, OCORₐ, ORₐ, NRₐR_{b}, CRₐ═NOR_{b}, NRₐCOR_{b}, NRₐCOOR_{b}, OCONRₐR_{b}, OCO₂Rₐ, NRₐCONR_{b}R_{c}, NRₐSO₂R_{b}, S(O)ₙRₐ, and SO₂NRₐR_{b}, CONRₐOR_{b}, N(OR_{b})CORₐ all the above members of the group representing W1 or W2 being optionally substituted by one, two or three identical or different groups R, which may form a cycle with each other;

Rₐ, R_{b} and R_{c}, identical or different, are selected from the group consisting of H, (C₁-C₆)alkyl, C₁-C₆)fluoroalkyl, (C₂-C₆)alkenyl, (C₂-C₆)alkynyl, phenyl, benzyl and 4-6 membered monocyclic saturated or unsaturated heterocycle containing 1-3 heteroatoms selected from N, O and S; Rₐ, R_{b} and R_{c} may form a cycle with each other optionally including 1-3 heteroatoms selected from N, O and S, illustrative examples of saturated nitrogen containing heterocycles within the definition of NRaRb include those selected from the group comprising, pyrrolidinyl, oxazolidinyl, thiazolidinyl, piperidinyl, piperazinyl and morpholinyl;

R is selected from the group consisting of halogen, CN, (C₁-C₆)alkyl, (C₁-C₆)fluoroalkyl, (C₂-C₆)alkenyl, (C₂-C₆)alkynyl, phenyl, 4-10 membered monocyclic or bicyclic saturated or unsaturated heterocycle containing 1-5 heteroatoms selected from N, O and S; CO₂Rₐ, CORₐ, CONRₐR_{b}, OCORₐ, ORₐ, NRₐR_{b}, CRₐ═NOR_{b}, NRₐCOR_{b}, NRₐCOOR_{b}, OCONRₐR_{b}, OCO₂Rₐ, NRₐCONR_{b}R_{c}, NRₐSO₂R_{b}, S(O)ₙRₐ, and SO₂NRₐR_{b}, CONRₐOR_{b}, N(OR_{b})CORₐ; all the above members of the group representing R being optionally substituted by one or several identical or different groups R′, which may form a cycle with each other;

R′ is selected from the group consisting of halogen, CN, (C₁-C₆)alkyl, (C₁-C₆)fluoroalkyl, (C₂-C₆)alkenyl, (C₂-C₆)alkynyl, phenyl, 4-10 membered monocyclic or bicyclic saturated or unsaturated heterocycle containing 1-5 heteroatoms selected from N, O and S; CO₂Rₐ, CORₐ, CONRₐR_{b}, OCORₐ, ORₐ, NRₐR_{b}, CRₐ═NOR_{b}, NRₐCOR_{b}, NRₐCOOR_{b}, OCONRₐR_{b}, OCO₂Rₐ, NRₐCONR_{b}R_{c}, NRₐSO₂R_{b}, S(O)ₙRₐ, and SO₂NRₐR_{b}, CONRₐOR_{b}, N(OR_{b})CORₐ;

n is 0, 1 or 2;

their N-oxide derivatives,
in their racemic, scalemic (non racemic mixtures), enantiomeric or geometric forms,
and their addition salts thereof with acids and bases;

to the exclusion of the following compounds:
Methyl (6R/S)—C-ethyl-α-D-gluco-pyranoside;
D/L- Glycero-D-manno-heptose-7-phosphate, and methyl α-D-manno-heptopyranoside-7-phosphate;
O-L-glycero-α-D-manno-heptopyranosyl-(1->7)-L-glycero-D-manno-heptopyranose;
Methy/7-O-L-glycero-α-D-manno-heptopyranosyl-L-glycero-α-D-manno-heptopyranoside;
Ally/6-O-(L-glycero-α-D-manno-heptopyranosyl)-α-D-gluco-pyranoside;
Methyl 7-O-(2-aminoethyl)phosphoryl-L-glycero-α-D-manno-heptopyranoside;
and with the proviso that when X is O and Y is H, W1 and W2 may not form a double bond with each other when W1 is a linking bond and W2 is (O)(D/L-Glycero-D-manno-hepto-1,5-pyranone); when W1 is H then W2 is different from OH, OCH₃, CH₂CH(CH₃)OH, CH₂C(O)CH₃, or when W2 is H then W1 is different from OH, OCH₃, OBn, OCH₂CH═CH₂, CH₂CH(CH₃)OH, CH₂C(O)CH₃, SC₂H₅, (N-Benzylcarbamoyl)-3-propyloxy, 3-(Perfluorooctyl)propanyl-oxybutanyloxy.

The above exclusions are disclosed in the following documents:
Methyl (6R/S)—C-ethyl-α-D-gluco-pyranoside:
Czernecki et al. J. Org. Chem. 1995, 60, 650-655; Spohr et al. Canadian J. Chem. 2001, 79, 238-256;
D/L-Glycero-D-manno-heptose-7-phosphate, and methyl α-D-manno-heptopyranoside-7-phosphate:
Grzeszczyk et al. Carbohydr. Res. 1998, 307, 55-68; Guezlek et al Carbohydrate Research, 2005, 340, 2808-2811;
O-L-glycero-α-D-manno-heptopyranosyl-(1->7)-L-: glycero-D-manno-heptopyranose
Holst et al. Carbohydr. Res. 1990, 204, 1-9: Dziewiszek et al. Tet. Lett. 1987, 28, 1569-1572;
Methy/7-O-L-glycero-α-D-manno-heptopyranosyl-L-glycero-α-D-manno-heptopyranoside:
Garegg et al. Carbohydr. Res. 1990, 205, 125-132;
Ally/6-O-(L-glycero-α-D-manno-heptopyranosyl)-α-D-glycero-pyranoside:
Nepogod'ev et al. Carbohydr. Res. 1994, 254, 43-60; Antonov et al. Carbohydr. Res. 1998, 314, 85-94);
Methyl 7-O-(2-aminoethyl)phosphoryl-L-glycero-α-D-manno-heptopyranoside:
Stewart et al. Carbohydr. Res. 1998, 313, 193-202; and
and with the proviso that when X is O and Y is H, W1 and W2 may not form a double bond with each other when W1 is a linking bond and W2 is (O)(D/L-Glycero-D-manno-hepto-1,5-pyranone); when W1 is H then W2 is different from OH, OCH₃, CH₂CH(CH₃)OH, CH₂C(O)CH₃, or when W2 is H then W1 is different from OH, OCH₃, OBn, OCH₂CH═CH₂, CH₂CH(CH₃)OH, CH₂C(O)CH₃, SC₂H₅, (N-Benzylcarbamoyl)-3-propyloxy, 3-(Perfluorooctyl)propanyl-oxybutanyloxy:
U.S. Pat. No. 5,798,343, 1998; Yamasaki et al. J. Carbohydr. Chem. 2001, 20, 171-180; Graziani et al. Tetrahedron: Asymm. 2005, 16, 167-176; Boons et al. Tetrahedron, 1992, 48, 885-904; Brimacombe et al. Carbohydr. Res. 1986, 152, 329-334; Palmelund et al. J. Org. Chem. 2005, 70, 8248-8251; Dasseret al. J. Chem. Soc., Perkin Trans. 1, 1990, 3091-3094; Khare et al. Canad. J. Chem. 1994, 72, 237-246; Jarrell et al. Canad. J. Chem. 1978, 56, 144; Reiter et al. Carbohydr. Res. 1999, 317, 39-52; Martin et al. Chem. Lett. 2004, 33, 696-697; Boons et al. Rec. Tray. Chim. Pays-Bas, 1989, 108, 339-343; Pohl et al. Angew. Chem. Int. Ed. 2008, 47, 1707-1710; Shaban et al. Carbohydrate Research, 1990, 203, 330-335.

According to a preferred embodiment, W1 and/or W2 is H, and X is O, S, CH₂ or NH, and Y is H, P(O)(OZ1)(OZ2) or P(O)(OZ1)(NHZ2).

According to another embodiment, carbon-6 is in D-glycero-heptose configuration. According to still another embodiment, in the above formula, X is O and Y is H. In still another embodiment, W1 and W2 are H. In other compounds, X is CH₂, CHF or CF₂ and Y is P(O)(OZ1)(OZ2).

Among the acid salts of the products of formula (I), there may be cited, among others, those formed with mineral acids, such as hydrochloric, hydrobromic, hydroiodic, sulfuric or phosphoric acid or with organic acids such as formic, acetic, trifluoroacetic, propionic, benzoic, maleic, fumaric, succinic, tartaric, citric, oxalic, glyoxylic, aspartic, alkanesulfonic acids, such as methanesulfonic and ethanesulfonic acids, arylsulfonic acids such as benzenesulfonic and para-toluenesulfonic acids. Among the alkaline salts of the products of formula (I), there may be cited, among others, those formed with mineral alkalis such as, for example, sodium, potassium, lithium, calcium, magnesium or ammonium or organic bases such as, for example, methylamine, ethylamine, propylamine, trimethylamine, diethylamine, triethylamine, N,N-dimethylethanolamine, tris(hydroxymethyl)aminomethane, ethanolamine, pyridine, piperidine, piperazine, picoline, dicyclohexylamine, morpholine, benzylamine, procaine, lysine, arginine, histidine, N-methylglucamine.

In the general formula (I), as applied herein:

“(C₁-C₆)alkyl” means any linear, branched or cyclic hydrocarbon groups having 1 to 6 carbon atoms, preferably methyl, ethyl, n-propyl, isopropyl, n-butyl, isobutyl and t-butyl, n-pentyl, isopentyl, neopentyl, cyclopropyl, cyclobutyl, cyclopentyl, cyclohexyl;

“(C₂-C₆)alkenyl” and “(C₂-C₆)alkynyl” as applied herein means any linear, branched or cyclic hydrocarbon groups of 2 to 6 carbon atoms, having at least one double bond or one triple bond and preferably ethenyl, propenyl, butenyl, cyclohexenyl, ethynyl, propargyl, butynyl;

“Halogen” means F, CI, Br, and I;

Illustrative heterocycles as mentioned in the definitions of formula I are for example those selected from the group comprising furyl, tetrahydrofuryl, benzofuryl, tetrahydrobenzofuryl, thienyl, tetrahydrothienyl, benzothienyl, tetrahydrobenzothienyl, pyrrolyl, pyrrolidinyl, indolyl, indolinyl, tetrahydroindolyl, oxazolyl, oxazolinyl, oxazolidinyl, benzoxazolyl, tetrahydrobenzoxazolyl, oxazolopyridinyl, tetrahydrooxazolopyridinyl, oxazolopyrimidinyl, tetrahydrooxazolopyrimidinyl, oxazolopyrazinyl, oxazolopyridazinyl, oxazolotriazinyl, isoxazolyl, benzoisoxazolyl, tetrahydrobenzoisoxazolyl, thiazolyl, thiazolinyl, thiazolidinyl, benzothiazolyl, tetrahydrobenzothiazolyl, thiazolopyridinyl, tetrahydrothiazolopyridinyl, thiazolopyrimidinyl, tetrahydrothiazolopyrimidinyl, thiazolopyrazinyl, thiazolopyridazinyl, thia-zolotriazinyl, isothiazolyl, benzoisothiazolyl, tetrahydrobenzoisothiazolyl, imi-dazolyl, benzimidazolyl, tetrahydrobenzimidazolyl, pyrazolyl, indazolyl, tetra-hydroindazolyl, triazolyl, oxadiazolyl, thiadiazolyl, tetrazolyl, pyranyl, dihydro-pyranyl, tetrahydropyranyl, benzopyranyl, dioxanyl, benzodioxanyl, dioxolanyl, benzodioxolanyl, pyridinyl, pyridonyl, piperidinyl, tetrahydropyridinyl, quinolinyl, isoquinolinyl, tetra- and perhydro-quinolinyl and isoquinolinyl, pyrimidinyl, quinazolinyl, pyrazinyl, pyrazidinyl, piperazinyl, quinoxalinyl, piridazinyl, cinno-linyl, phtalazinyl, triazinyl, purinyl, pyrazolopyridinyl, tetrahydropyrazolopyridinyl, pyrazolopyrimidinyl, pyrazolopyrazinyl, pyrazolotriazinyl, triazolopyridinyl, tetra-hydrotriazolopyridinyl, triazolopyrimidinyl, triazolopyrazinyl, triazolotriazinyl, oxetanyl, azetidinyl, morpholinyl.

Compounds of formula I may be prepared by any processes known to be applicable to the preparation of chemically related compounds (for a review example: Curr. Org. Chem. 2008, 1021). Such processes may use known starting materials or intermediates which may be obtained by standard procedures of organic chemistry. The following processes provide a variety of non-limiting routes for the production of the compounds of formula I and their intermediates.

Examples of processes to prepare compounds of formula (I) and salts thereof include in non-limiting manner: the transformation of compounds of formula (II) into compounds of formula (I)

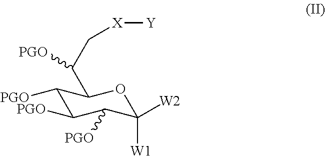

wherein X, Y, W1 and W2 are as above defined, X, Y, W1 and W2 optionally protected by one or several identical or different protecting group PG;

PG is H or an appropriate identical or different protecting group (non-limiting examples include optionally substituted benzyl, silyl groups, acyl);

by one or more of the non-limiting appropriate following reactions, performed in an appropriate order, to achieve the transformations on W and/or Y and/or PG defined above:

protection of reactive functions,

deprotection of reactive functions,

halogenation,

dehalogenation,

dealkylation,

alkylation of amine, aniline, alcohol and phenol,

oxidation,

Wittig type reaction on carbonyl groups,

dihydroxylation reaction of carbon-carbon double bonds,

reduction of nitro, esters, cyano, aldehydes, thioethers, double and triple

bonds,

transition metal-catalyzed reactions,

etherification,

acylation,

sulfonylation/introduction of sulfonyl groups,

saponification/hydrolysis of esters groups,

halogen exchange,

nucleophilic substitution with amine, thiol or alcohol,

reductive amination,

phosphorylation,

sulphatation,

phosphitation,

phosphonylation,

amidation,

phosphoramidation,

fixation of R and/or R′ and/or Ra and/or Rb and/or Rc groups on W1 or W2

fixation of groups Z1 and/or Z2 on Y,

salification;

all of these reactions optionally followed by deprotection of PG to hydrogen.

Compounds of formula I and salts thereof may also be prepared in non-limiting manner by transformation at the anomeric position of compounds of formula (III), or a salt thereof:

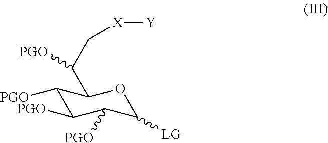

wherein X and Y are as above defined, optionally protected by one or several identical or different protecting group PG;

PG is H or an appropriate identical or different protecting group (non-limiting examples include optionally substituted benzyl, silyl groups, acyl); LG is an appropriate leaving group (non-limiting examples include hydroxyl, thioaryl, O-acyl, halogen, phosphonium, sulfonyloxy, NRₐR_{b} or ORₐ).

Displacement of the leaving group at the anomeric position of compounds of formula (III) occurs by optional leaving group activation with an halogenated reagent (non-limiting example include NCS or NBS in the case of thioaryl), following nucleophilic substitution with any appropriate nucleophile (non-limiting examples include allyltrimethylsilane with appropriate Lewis acid(s) in the case of allylation of acetate leaving group, or with DAST in the case of the fluoration of the hydroxyl leaving group), following with optional hydrolysis, alkylation, acylation, reduction, oxidation, substitution, optionally followed by deprotection of PG to hydrogen.

Compounds of formula (I) and salts thereof may also be prepared in non-limiting manner by transformation at the anomeric oxygen from compounds of formula (IV), or a salt thereof:

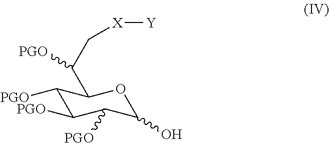

wherein X and Y are as above defined, optionally protected by one or several identical or different protecting group PG;

PG is H or an appropriate identical or different protecting group (non-limiting examples include optionally substituted benzyl, silyl groups, acyl);

Nucleophilic substitution by the anomeric hydroxyl with any appropriate electrophilic reacting groups optionally attached to a leaving group LG as defined above may achieve the desired transformation (non-limiting example includes iodomethane with appropriate base like silver oxide in the case of a methylation), optionally followed by deprotection of PG to hydrogen.

Compounds of formula (I) and salts thereof or intermediates of the synthetic route towards compounds of formula (I) may also be obtained in non-limiting manner by transformation at position 7 of compounds of formula (V) or a salt thereof, by reacting a compound of formula (V):

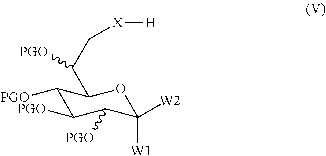

with Y-LG,

X, Y, LG, W1 and W2 defined as above with X, Y, W1 and W2 optionally protected by one or several identical or different protecting groups PG, PG is H or an appropriate identical or different protecting group (non-limiting examples include optionally substituted benzyl, silyl groups, acyl);

(non-limiting example includes phosphorylation with (RₐO)(R_{b}O)P(O)-LG, such as nucleophilic substitution in case LG is halogen, or Mitsunobu reaction in case LG is hydroxy), following by optional oxidation (non-limiting example includes mCPBA oxidation of phosphite to phosphate derivatives), optionally followed by deprotection of PG to hydrogen.

Compounds of formula (I) and salts thereof or intermediates of the synthetic route towards compounds of formula (I) may also be obtained in non-limiting manner by transformation at position 7 of compounds of formula (VI) or a salt thereof, by reacting a compound of formula (VI):

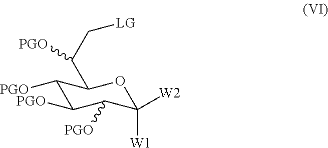

with MeP(O)(OZ1)(OZ2), CH₂FP(O)(OZ1)(OZ2) or CHF₂P(O)(OZ1)(OZ2), in the presence of a suitable base,

X, Y, LG, W1 and W2 defined as above with X, Y, W1 and W2 optionally protected by one or several identical or different protecting groups PG, PG is H or an appropriate identical or different protecting group (non-limiting examples include optionally substituted benzyl, silyl groups, acyl);

(non-limiting example includes methylphosphonylation, fluoromethylphosphonylation or difluoromethylphosphonylation with bases such as BuLi or LDA), optionally followed by deprotection of PG to hydrogen.

Heptoses of formula (I), salts thereof, and heptose intermediates of the synthetic route towards compounds of formula (I) can also be obtained by homologation of corresponding hexoses according to known processes (J. Org. Chem. 2000, 65, 6493; Chem. Eur. J. 2008, 14, 9530; Pol. J. Chem. 1996, 70, 45; Angew. Chem. 2008, 120, 1731; Carbohydr. Res. 2005, 340, 2808; Carbohydr. Res. 1986, 152, 329; J. Am. Chem. Soc. 2006, 128, 8078).

Compounds of formula (I) are capable of inhibiting bacterial heptose synthesis which makes them useful as drugs for preventing or treating bacterial infections and another object of the invention is the use of the compounds of formula (I) as drugs. The drugs of the invention are especially useful for the prevention and therapeutical treatment of severe infections due to Gram-negative bacteria able to disseminate in blood such as the non-limiting following species (spp.): Escherichia coli, Enterobacter, Salmonella, Shigella, Pseudomonas, Acinetobacter, Neisseria, Klebsiella, Serratia, Citrobacter, Proteus, Yersinia, Haemophilus, Legionella, Moraxella and Helicobacter pylori.

The invention also relates to pharmaceutical compositions comprising an effective amount of at least one compound of formula (I) such as above defined, in association with a pharmaceutically acceptable carrier. The pharmaceutical compositions are advantageously formulated to be administered under oral, parenteral, and preferably injectable routes, with individual doses appropriate for the patient to be treated.

The compositions according to the invention can be solid or liquid and be present in the pharmaceutical forms commonly used in human medicine, such as for example, plain or sugar-coated tablets, gelatin capsules, granules, suppositories, inhalation spray, injectable preparations, ointments, creams, gels; they are prepared according to the customary methods. The active ingredient(s) can be incorporated in same, using excipients which are customarily used in these pharmaceutical compositions, such as talc, gum arabic, lactose, starch, magnesium stearate, cocoa butter, aqueous or non-aqueous vehicles, fatty substances of animal or vegetable origin, paraffin derivatives, glycols, various wetting agents, dispersants or emulsifiers, preservatives. These compositions can in particular be present in the form of a powder intended to be dissolved extemporaneously in an appropriate vehicle, for example, non-pyrogenic sterile water.

The dose administered varies according to the condition treated, the patient in question, the administration route and the product envisaged. It can, for example, be comprised between 0.1 g and 10 g per day, by oral route in humans or by intramuscular or intravenous route. The drugs according to the invention can also be advantageously combined with other antibacterials.

A further object of the invention is therefore the associations of the compounds of formula (I) with antimicrobial peptides or natural, hemisynthetic or synthetic antibacterial molecules as well as pharmaceutical compositions containing them. Other characteristics and advantages of the invention are given in the following examples.

### BRIEF DESCRIPTION OF THE DRAWING

FIG. 1 is a scan obtained with a gel electrophoresis.

### DETAILED DESCRIPTION

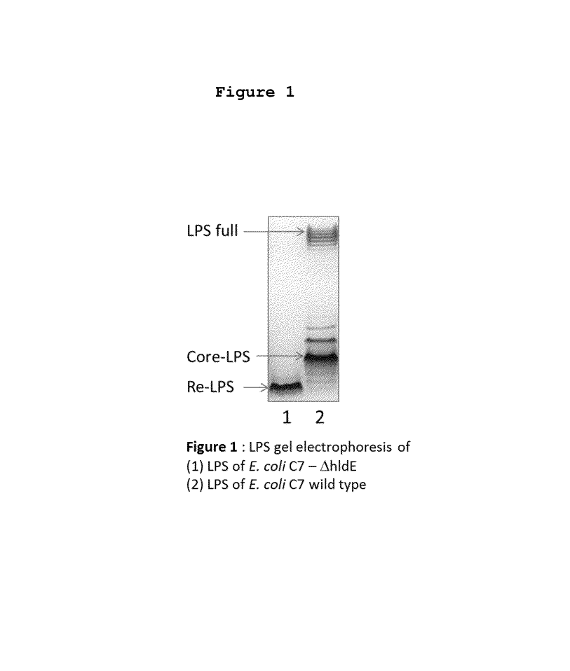

In the results concerning the pharmacological study of the compounds of the invention, it is referred to FIG. 1, which provides positive and negative controls obtained with a gel electrophoresis of (1) LPS of E. coli C7-ΔhldE and (2) LPS of E. coli C7 wild type.

Experimental Part

Materials and Procedures

All chemicals were purchased from Sigma, Aldrich or Fluka and were used without further purification. Tetrahydrofuran, diethyl ether and toluene were freshly distilled over sodium benzophenone, dichloromethane over P₂O₅ and acetonitrile over CaH₂. ¹H—, ¹³C— and ³¹P-NMR spectra were recorded with JEOL 270 and 400 MHz spectrometers. All compounds were characterized by ¹H— (chemical shifts are reported in parts per million downfield from the internal standard tetramethylsilane), ¹³C— and ³¹ P-NMR as well as by ¹H—¹H and ¹H—¹³C correlation experiments. Abbreviations for NMR data are as follows: s=singlet, d=doublet, t=triplet, q=quadruplet, se=sextuplet, m=multiplet, dd=doublet of doublets, dt=doublet of triplets, ddd=doublet of doublets of doublets, br=broad, Cq=quaternary carbon. J indicates the NMR coupling constant measured in Hertz. Specific optical rotations were measured on a Perkin Elmer 241 Polarimeter in a 1 dm cell. Melting points were determined with a Büchi 535 apparatus. Column chromatographies were performed on silica gel Kieselgel Si 60 (40-63 μm). When required, purifications were realized by semi-preparative HPLC, using a Waters Delta prep 4000 chromatography system equipped with a Zorbax C18-SB (Agilent) or a GRACE-C18 semi-prep column. Analytical chromatograms were recorded on a Waters 600 E apparatus equipped with the corresponding Zorbax C18-SB or GRACE-C18 columns (25*0.46 cm, 5 μm). LC-MS measurements were performed on a Agilent 6200 series TOF mass spectrometer operating in positive mode. The analyte solutions were delivered to the ESI source by a Agilent 1200 series LC system at a flow rate of 0.25 mL/min. Typical elution gradient start from water (90%) to acetonitrile (100%) with optional additional 0.1% formic acid for 20 minutes. Typical ESI conditions were: capillary voltage, 2.0 kV; cone voltage, 65 V; source temperature, 150° C.; desolvation temperature, 250° C. drying gas: 51/min, nebuliser 60 psig.

Typical APCI condition were: capillary voltage, 2.0 kV; cone voltage, 65 V; source temperature, 250° C.; desolvation temperature, 350° C. drying gas: 51/min, nebuliser 60 psig. Dry nitrogen was used as the ESI and APCI gas. For the recording of the single-stage ESI-MS spectra all ions were transmitted into the pusher region of the time-of-flight analyzer where they were mass analyzed with 1 s integration time. HRMS were obtained with a JMS-700 spectrometer.

The meaning of certain abbreviations is given herein. D₂O is deuterated water, CDCl₃ is deuteriochloroform, DMSO-d₆ is hexadeuteriodimethylsulfoxide, and CD₃OD is tetradeuteriomethanol. LC refers to liquid chromatography, MS refers to mass spectrometry, HRMS refers to high resolution mass spectrometry, ESI refers to electrospray ionization, TOF-MS refers to time-of-flight mass spectrometry, HPLC refers to high pressure liquid chromatography, M in the context of mass spectrometry refers to the molecular peak, NMR refers to nuclear magnetic resonance, NOE refers to nuclear overhauser effect, pH refers to potential of hydrogen, TLC refers to thin layer chromatography, THF refers to tetrahydrofuran, DMF refers to N,N-dimethylformamide, DCM refers to dichloromethane, DMSO refers to dimethylsulfoxide, TIPSCI refers to triisopropylsilylchloride, TBAF refers to tetra-n-butyl ammonium fluoride, TEA refers to triethylamine, NBS refers to N-bromosuccinimide, NCS refers to N-chlorosuccinimide, PCC refers to pyridinium chlorochromate, DAST refers to diethylaminosulfur trifluoride, DEAD refers to diethylazodicarboxylate, TMS refers to trimethylsilyl, 4-DMAP refers to 4-dimethylaminopyridine, TMSOTf refers to trimethylsilyltriflate.

### Example 1

### Methyl 7-O-phosphate-D-glycero-α-D-gluco-heptopyranoside

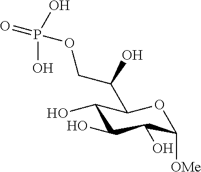

### Step 1: Methyl 2,3,4-tri-O-benzyl-D/L-glycero-α-D-gluco-heptopyranoside

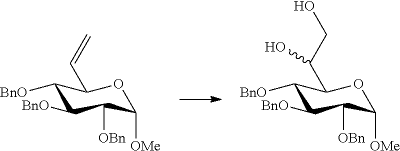

To a solution of methyl 6,7-dideoxy-2,3,4-tri-O-benzyl-glycero-α-D-gluco-hept-6-eno-pyranoside prepared according to A. M. Aurrecoechea, B. Lopez, M. Arrate, J. Org. Chem. 2000, 65, 6493-6501. b. Dohi, H.; Perion, R.; Durka, M.; Bosco, M.; Roué, Y.; Moreau, F.; Grizot, S.; Ducruix, A.; Escaich, S.; Vincent, S. P. Chem. Eur. J. 2008, 14, 9530-9539 (800 mg, 1.73 mmol, 1 eq.) and N-methyl morpholine oxide (326 mg, 2.78 mmol, 1.6 eq.) in a mixture of acetone/H₂O 2:1 (8.0 mL) at room temperature, was added K₂OsO₄.2(H₂O) (60 mg, 0.17 mmol, 0.1 eq.) in one portion. After 15 hours, a saturated solution of Na₂SO₃ (20 mL) was added. The reaction mixture was extracted with EtOAc (3×20 mL). The organic phase was washed with an aqueous solution of HCl 4% (20 mL), with a saturated solution of NaHCO₃ (20 mL), brine (20 mL), and was dried over MgSO₄, filtered and concentrated under reduced pressure. Purification of the residue by flash chromatography (SiO₂, Cyclohexane/EtOAc 9:1 to Cyclohexane/EtOAc 7:3) afforded a mixture of D- and L-diol derivatives (700 mg, 82%) as a colorless oil. The D/L ratio of 8:2 was determined by ¹H NMR. The molecule has been previously described in the literature (S, Jarosz, E. Koziowska, Pol. J. Chem, 1996, 70, 45-53),

### Step 2: Methyl 2,3,4-tri-O-benzyl-7-O-triisopropylsilyl-D/L-glycero-α-D-gluco-heptopyranoside

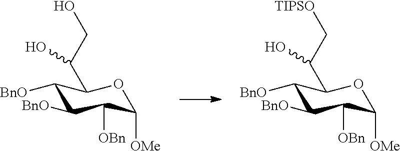

To a mixture of methyl 2,3,4-tri-O-benzyl-D/L-glycero-α-D-gluco-heptopyranoside (700 mg, 1.40 mmol, 1 eq.) and imidazole (286 mg, 4.20 mmol, 3 eq.) in dry THF (14 mL) under Argon atmosphere, was added dropwise at 0° C., TIPSCI (600 μL, 2.80 mmol, 2 eq.) The reaction was then allowed to warm to room temperature and was stirred for 15 hours. The reaction was quenched by addition of an aqueous solution of saturated NH₄Cl (50 mL) and extracted with DCM (3×30 mL). The organic layer was dried over MgSO₄, filtered and concentrated in vacuo. Purification of the residue by flash chromatography (SiO₂, Cyclohexane/EtOAc 9:1 to Cyclohexane/EtOAc 7:3) afforded a D/L mixture of silylated derivatives (790 mg, 87%) as a colorless oil.

Step 3:

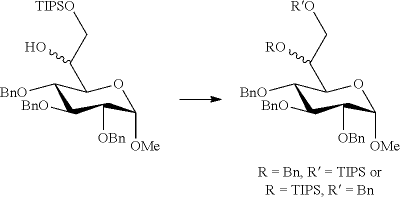

To a cold solution (0° C.) of the previous D/L mixture (700 mg, 1.1 mmol, 1 eq.) in dry DMF (15 mL) under argon atmosphere was added dropwise benzyl bromide (280 μL, 2.9 mmol, 2.6 eq.) and, with care, in portions, sodium hydride (60% in oil, 90 mg, 2.26 mmol, 2.1 eq.). The reaction mixture was allowed to warm to room temperature after the addition. The reaction was quenched after 15 hours by a slow addition of methanol (2 mL), followed by cold water (50 mL). The reaction was extracted with Et₂O (3×30 mL), washed with brine (2×10 mL), dried over MgSO₄, filtered and concentrated under reduced pressure. Purification of the residue by flash chromatography (SiO₂, Cyclohexane/EtOAc 100:0 to 90:10) afforded a mixture of derivatives (750 mg, 94%) as a colorless oil. This mixture was engaged in the next step for characterization.

### Step 4: Methyl 2,3,4,6-tetra-O-benzyl-D-glycero-α-D-gluco-heptopyranoside

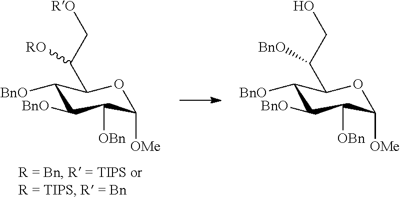

To a solution of the previous mixture (750 mg, 1.01 mmol, 1 eq.) in dry THF (20 mL) under argon atmosphere was added TBAF.3 H₂O (380 mg, 1.21 mmol, 1.2 eq.). The reaction mixture was stirred for 15 hours at room temperature. The mixture was diluted with a saturated solution of NH₄Cl (20 mL) and extracted with DCM (3×15 mL). The organic phase was washed with water (10 mL) and brine (10 mL), dried over MgSO₄, filtered and concentrated under reduced pressure. Purification of the residue by flash chromatography (SiO₂, Cyclohexane/EtOAc 8:2 to 7:3) afforded the desired derivative (435 mg, 73%) as a colorless oil.

¹H NMR (400 MHz, CDCl₃) δ (ppm) 7.37-7.17 (m, 20H, H^{arom}), 5.00 (d, J=10.8 Hz, 1H, CH₂^{Bn}), 4.90 (d, J=11.0 Hz, 1H, CH₂^{Bn}), 4.81 (d, J=6.9 Hz, 1H, CH₂^{Bn}), 4.78 (d, J=8.5 Hz, 1H, CH₂^{Bn}), 4.71 (d, J=11.7 Hz, 1H, CH₂^{Bn}), 4.66 (d, J=12.1 Hz, 1H, CH₂^{Bn}), 4.60 (d, J=8.9 Hz, 1H, CH₂^{Bn}), 4.59 (d, J=1.4 Hz, 1H, H-1), 4.55 (d, J=11.9 Hz, 1H, CH₂^{Bn}), 4.00 (t, J=9.2 Hz, 1H, H-4), 3.94 (dd, J=10.3 Hz, J=0.9 Hz, 1H, H-5), 3.74 (ddd, J=1.1 Hz, J=3.9 Hz, J=6.6 Hz, 1H, H-6), 3.68 (m, 1H, H-7a), 3.56 (m, 1H, H-7b), 3.47 (m, 2H, H-2H-3), 3.38 (s, 3H, H^{Me}), 1.98 (d, J=6.4 Hz, 1H, OH).

¹³C NMR (100 MHz, CDCl₃) δ (ppm) 138.6 138.2 138.1 137.9 (4C_{q}^{arom}), 128.6-127.8 (20CH^{arom}), 97.8 (C-1), 82.6 (C-4), 80.1 (C-3), 78.6 (C-6), 77.6 (C-2), 76.0 74.9 73.5 72.2 (4CH₂^{Bn}), 70.7 (C-5), 61.7 (C-7), 55.3 (C^{Me}).

### Step 5: Methyl 7-O-dibenzylphosphate-2,3,4,6-tetra-O-benzyl-D-glycero-α-D-gluco-heptopyranoside

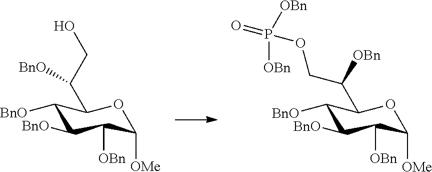

To the previous alcohol (250 mg, 0.43 mmol, 1 eq.), PPh₃ (485 mg, 1.85 mmol, 4.3 eq.), dibenzylphosphate (515 mg, 1.85 mmol, 4.3 eq.), and NEt₃ (500 μL, 3.60 mmol) in solution in distilled THF (3.5 mL), was slowly added diethyl azodicarboxylate 40% in solution in toluene (820 μL, 1.85 mmol, 4.3 eq.) under argon atmosphere at room temperature. The reaction mixture was light yellow. After 15 hours, the solvent was removed in vacuo. Purification of the residue by flash chromatography (SiO₂, Cyclohexane/EtOAc 8:2 to 7:3) afforded the desired product (360 mg, 97%).

[α]_{d}²⁰(CHCl₃, c=1)=+19.8°.

³¹P NMR (101 MHz, CDCl₃) δ (ppm)−0.29.

¹H NMR (400 MHz, CDCl₃) δ (ppm) 7.38-7.17 (m, 30H, H^{arom}), 5.01-4.91 (m, 5H, CH₂^{Bn}), 4.84 (d, J=11.0 Hz, 1H, CH₂^{Bn}), 4.77 (d, J=10.8 Hz, 1H, CH₂^{Bn}), 4.76 (d, J=10.5 Hz, 1H, CH₂^{Bn}), 4.64 (d, J=10.9 Hz, 2H, CH₂^{Bn}), 4.55 (d, J=11.7 Hz, 2H, CH₂^{Bn}), 4.54 (d, J=3.7 Hz, 1H, H-1), 4.15 (m, 2H, H-7), 3.96 (t, J=9.2 Hz, 1H, H-3), 3.91 (m, 1H, H-6), 3.85 (d, J=10.5 Hz, 1H, H-5), 3.50 (dd, J=10.3 Hz, J=8.9 Hz, 1H, H-4), 3.43 (dd, J=9.9 Hz, J=3.7 Hz, 1H, H-2), 3.34 (s, 3H, H^{Me}).

¹³C NMR (100 MHz, CDCl₃) δ (ppm) 138.7-135.8 (6C_{q}^{arom}), 128.6-127.7 (30CH^{arom}), 97.9 (C-1), 82.4 (C-3), 80.0 (C-2), 78.1 (d, J=7.7 Hz, C-6), 77.9 (C-4), 75.9 74.9 73.4 72.9 (4CH₂^{Bn}), 70.5 (C-5), 69.3 (appt, J=4.8 Hz, 2CH₂^{Bn}), 67.6 (d, J=5.8 Hz, C-7), 55.3 (C^{Me}).

MS (ESI⁺): m/z: 867.33(100%) [M+Na]⁺, 607.27(60%) [C₃₆H₄₀O₇+Na]⁺, 862.37 (60%) [M+NH₄]⁺, 279.09 (20%) [C₁₄H₁₅O₄P+H]⁺.

HRMS (TOF-MS-ESI⁺): calculated for C₅₀H₅₃O₁₀PNa, [M+Na]⁺: 867.3269. found: 867.3265.

Elemental analysis: calculated (%) for C₅₀H₅₃O₁₀P:C 71.08, H 6.32. found: C 70.62, H 6.64.

### Step 6: Methyl 7-O-phosphate-D-glycero-α-D-gluco-heptopyranoside

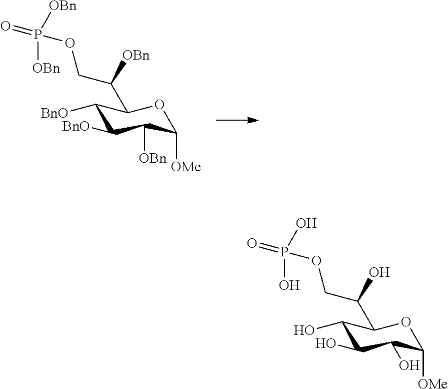

To the previous phosphate derivative (85 mg, 0.1 mmol, 1 eq.) in solution in EtOAc/EtOH/H₂O 3:5:2 (3.5 mL) under Argon atmosphere was added in one portion Pd/C 10% (80 mg, 75 μmol, 0.75 eq.). The reaction mixture was stirred vigourously under hydrogen atmosphere for 2 hours (a TLC in Cyclohexane/EtOAc 1:1 showed total conversion). The reaction mixture was degassed and filtered on a celite pad. The celite pad was washed with the reaction solvent mixture (5 mL) and the filtrate was reduced in vacuo (bath temperature 27° C.) and lyophilized to afford the desired unprotected heptoside (36 mg, 100%) as a white foam.

[α]_{d}²⁰(CH₃OH, c=0.5)=+23.8°.

³¹P NMR (101 MHz, D₂O) δ (ppm) 1.01.

¹H NMR (400 MHz, D₂O) δ (ppm) 4.64 (d, J=3.7 Hz, 1H, H-1), 4.03 (m, 1H, H-6), 3.95 (m, 1H, H-7a), 3.82 (m, 1H, H-7b), 3.59 (dd, J=5.0 Hz, J=10.1 Hz, 1H, H-3), 3.49 (t, J=9.4 Hz, 1H, H-4), 3.42 (m, 1H, H-2), 3.38 (m, 1H, H-5), 3.27 (s, 3H, H^{Me}).

¹³C NMR (100 MHz, D₂O) δ (ppm) 99.2 (C-1), 73.3 (C-4), 71.5 (C-3), 71.0 (C-2), 70.5 (d, J=7.7 Hz, C-6), 70.1 (C-5), 65.7 (d, J=4.8 Hz, C-7), 55.1 (C^{Me}).

MS (ESI⁻): m/z: 303.0477 (100%) [M−H]⁻. HRMS (TOF-MS-ESI⁻): calculated for C₈H₁₆O₁₀P [M−H]⁻: 303.0487, experimental: 303.0477.

### Example 2

### Methyl 7-O-phosphoryl-D-glycero-α-D-manno-heptopyranoside

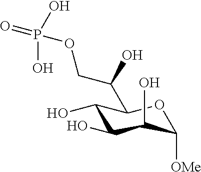

### Step 1: Phenyl-2,3,4,6-tetra-O-acetyl-1-thio-α-D-manno-pyranoside

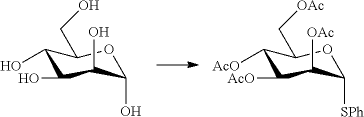

To a solution of commercially available D-mannose (50 g, 278 mmol, 1 éq.) in dry pyridine (250 mL) at 0° C. under argon atmosphere was added dropwise Ac₂O (175 mL, 1.85 mol, 6.7 eq.) in 1h20. The reaction mixture was allowed to warm to room temperature. After 15 h at room temperature and concentration in vacuo, the residue was diluted with dichloromethane (200 mL), washed with a solution of HCl (1M, 250 mL), a saturated aqueous solution of NaHCO₃ (250 mL) and water (250 mL), dried over MgSO₄, filtered, and concentrated in vacuo.

To a solution of the previous per-acetate (278 mmol, 1 eq.) in distilled dichloromethane (250 mL) under argon atmosphere were added thiophenol (42 mL, 416 mmol, 1.5 eq.) followed by Et₂O.BF₃ (176 mL, 1.39 mol, 5 eq.) in 0h45. After 15 h at room temperature, the reaction mixture was cooled to 0° C. and a saturated aqueous solution of NaHCO₃ (500 mL) was added. The reaction mixture was allowed to warm to room temperature, a saturated aqueous solution of NaHCO₃ (200 mL) was added and neutralisation was completed with cautious introduction of solid NaHCO₃ (68 g). The organic phase was then washed with water (250 mL), dried over MgSO₄, filtered and concentrated in vacuo. The residue was solubilised in a mixture of cyclohexane and diethyl ether and the solution was cooled to 0° C. as to induce a crystallisation. Filtration and washing with diethyl ether (2×70 mL) afforded the desired thio-mannoside (87 g, 71%) as white crystals.

m.p. 83.4-84° C.

[α]ₚ+114.7 (c1.00, CHCl₃).

¹H-NMR (400 MHz, CDCl₃): δ (ppm) 7.56-7.41 (m, 2H; Ph), 7.38-7.32 (m, 3H; Ph), 5.54-5.51 (m, 1H; H-2), 5.52 (bs, 1H; H-1), 5.40-5.30 (m, 2H; H-3, H-4), 4.57 (ddd, J_{4,5}=9.7 Hz, J_{5,6a}=2.4 Hz, J_{5,6b}=5.9 Hz, 1H; H-5), 4.33 (ABX, J_{5.6b}=5.9 Hz, H_{6a,6b}=12.2 Hz, 1H; H-6b), (ABX, J_{5,6a}=2.4 Hz, J_{6a,6b}=12.2 Hz, 1H; H-6a), 2.17 (s, 3H; COCH₃), 2.10 (s, 3H; COCH₃), 2.07 (s, 3H; COCH₃), 2.04 (s, 3H; COCH₃).

¹³C-NMR (100 MHz, CDCl₃): δ (ppm) 170.4, 169.8, 169.7, 169.6 (CH₃CO), 132.5 (Cq, Ph), 132.0, 129.1, 128.0 (CH, Ph), 85.6 (C-1), 70.8 (C-2), 69.4 (C-5), 69.3 (C-3), 66.3 (C-4), 62.3 (C-6), 20.8, 20.6, 20.6, 20.5 (CO CH₃).

¹H-non-decoupled ¹³C-NMR (100 MHz, CDCl₃): J_{C1-H1}=170 Hz.

MS (DCl—NH₃): m/z 548 (100%) [M+NH₄⁺], 331 (83%).

HRMS calculated for C₂₀H₂₈NO₉S: 458.1485 experimental 458.1491.

### Step 2: Phenyl-2,3,4-tri-O-benzyl-1-thio-α-D-manno-pyranoside

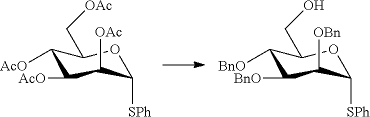

To a solution of the previous tetracetate (25 g, 56.7 mmol, 1 eq.) in anhydrous methanol (17 mL) under argon atmosphere, was added Na (20 mg, 0.9 mmol, 0.02 eq.). After 2 h, IR-120 (Amberlite H⁺ form) was added. The reaction mixture was filtered and the filtrate was concentrated in vacuo to afford the tetra-ol (15.5 g, quantitative), which was used without further purification.

To a solution of the previous tetra-ol (14.7 g, 54.1 mmol, 1 eq.) and imidazole (11 g, 162.2 mmol, 3 eq.) in distilled THF (108 mL) at 0° C. and under argon atmosphere, was added dropwise in 0h15 TIPSCI (12.2 mL, 56.8 mmol, 1.05 eq.). After 0h05 at 0° C., the reaction mixture was allowed to warm to room temperature and after 3h10, TIPSCI was added (0.6 mL, 2.7 mmol, 0.05 eq.). After 15 h, the reaction mixture was diluted with an aqueous saturated solution of NH₄Cl (100 mL), extracted with dichloromethane (4×100 mL). The organic phase was washed with brine (150 mL), dried over MgSO₄, filtered, concentrated in vacuo, and the residue was used without further purification.

To a solution of the previous triol (54.1 mmol, 1 eq.) and BnBr (23.1 mL, 194.6 mmol, 3.6 eq.) in anhydrous DMF (300 mL) at 0° C. and under argon atmosphere was added NaH (60% in mass, 13 g, 324 mmol, 6 eq.) in 5 times every 0h20. After 0h15 at 0° C., the solution was allowed to warm to room temperature. After 1 h05 at room temperature, the reaction mixture was cooled to 10° C., methanol (25 mL) was added dropwise and the mixture was warmed to room temperature. After concentration in vacuo, dichloromethane (150 mL) and water (200 mL) were successively added and the aqueous layer was extracted with dichloromethane (3×150 mL). The organic phase was washed with water (250 mL) and brine (250 mL), dried over MgSO₄, filtered, concentrated in vacuo, and the residue was used without further purification.

To a solution of the previous silylated ether (54.1 mmol, 1 eq.) in distilled THF (300 mL) under argon atmosphere was added TBAF.3H₂O (24.1 g, 108.1 mmol, 2 eq.). After 1h35, TBAF.3H₂O (1.7 g, 5.4 mmol, 0.1 eq.) was added. After 0h30, an aqueous saturated solution of NH₄Cl (200 ml) and dichloromethane (200 mL) were successively added and the aqueous phase was extracted with dichloromethane (3×150 mL). The organic phase was washed with water (250 mL) and brine (250 mL), dried over MgSO₄, filtered, concentrated in vacuo. Purification of the residue by column chromatography (silica gel 5-35 μm, 570 g, cyclohexane/EtOAc, 9:1 to 825:175) afforded the desired tri-benzylated mannoside (27.3 g, 50.3 mmol, 93%) as a colorless oil.

[α]D+92.2 (c 0.615, CHCl₃).

¹H-NMR (400 MHz, CDCl₃): δ (ppm) 7.49-7.27 (m, 20H; Ph), 5.59 (d, J_{1,2}=1.7 Hz, 1H; H-1), 5.03 (AB, J_{AB}=10.9 Hz, 1H; CH₂Ph), 4.80-4.72 (m, 3H; CH₂Ph), 4.73 (AB, J_{AB}=11.7 Hz, 1H; CH₂Ph), 4.68 (AB, J_{AB}=11.7 Hz, 1H; CH₂Ph), 4.20 (ddd, J_{4,5}=9.5 Hz, J_{5,6a}=3.8 Hz, J_{5,6b}=3.2 Hz, 1H; H-5), 4.13 (dd, J_{3,4}=9.2 Hz, J_{4,5}=9.5 Hz, 1H; H-4), 4.07 (dd, J_{1,2}=1.7 Hz, J_{2,3}=3.0 Hz, 1H; H-2), 3.97 (dd, J_{2,3}=3.0 Hz, J_{3,4}=9.2 Hz, 1H; H-3), 3.83 (m, 2H; H-6a, H-6b), 2.00 (bt, J=4.5 Hz, 1H; OH).

¹³C-NMR (100 MHz, CDCl₃) δ (ppm) 138.2, 138.0, 137.8 (Cq, Ph), 133.9 (Cq, SPh), 131.8, 129.0, 128.4, 128.0, 127.9, 127.8, 127.7, 127.7, 127.6 (CH, Ph), 86.0 (C-1), 80.0 (C-3), 76.3 (C-2), 75.2 (CH₂Ph), 74.7 (C-4), 73.2 (C-5), 72.3, 72.2 (CH₂Ph), 62.1 (C-6).

¹H-non-decoupled ¹³C-NMR (100 MHz, CDCl₃) J_{C1-H1}=167 Hz.

MS (FAB+, MB, NaI): m/z 565 (76%) [M+Na]⁺.

HRMS calculated for C₃₃H₃₄O₅SNa: 565.2025 experimental 565.2021.

### Step 3: Phenyl 6,7-dideoxy-2,3,4-tri-O-benzyl-1-thio-glycero-α-D-manno-hept-6-enopyranoside

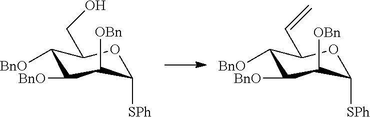

To a solution of the oxalylchloride (2.24 mL, 25.80 mmol) in distilled dichloromethane (129 mL) at −70° C. under argon atmosphere was added dropwise during 10 min DMSO (2.09 mL, 29.49 mmol). After 10 min, a solution of the primary alcohol (10 g, 25.80 mmol) in distilled dichloromethane (46 mL) was added dropwise. The reaction mixture stirred at the same temperature for one hour and the TEA (7.77 mL) was added dropwise during 10 min, and the mixture stirred for 5 min more. After that, the reaction warmed up to room temperature and stirred for one hour more, then an aqueous solution of NaHCO₃ (100 mL) was added, the organic phase was washed with water (100 mL), dried over MgSO₄, filtered, concentrated in vacuo, co-evaporated with toluene (3×10 mL) and the residue was used without purification.

To a suspension of methyltriphenylphosphonium bromide (17.92 g, 50.17 mmol) in distilled THF (67 mL) at 0° C. under argon atmosphere was quickly added n-BuLi (2.5 M in hexanes, 19.27 mL, 48.18 mmol) and the mixture was stirred for one hour at room temperature. Then, a solution of the aldehyde (10.84 g, 20.08 mmol) in dry THF (20 mL) was added dropwise at −78° C., then the reaction was allowed to warm up to room temperature and was stirred overnight. After that, aqueous saturated solution of NH₄Cl (100 mL) was added and the mixture was extracted with diethylether (5×150 mL). The organic phase was dried over MgSO₄, filtered, concentrated in vacuo and the residue was purified by silica gel chromatography eluted with (cyclohexane/ethyl acetate: 95/5) to afford the corresponding alkene (8.11 g, 75%) as white crystals.

mp 45.0-46.0° C.

[α]_{D}+100.3 (c0.98, CHCl3).

MS (CDl—NH₃) m/z 556 (100%) [M+NH₄]⁺.

HRMS calculated for C34H38NO4S: 556.2522 experimental: 556.2532.

¹H NMR (400 MHz, CDCl₃) δ (ppm) 7.48-7.25 (m, 20H, Ph), 6.09 (ddd, J_{6,7cis}=17.3 Hz, J_{6,7trans}=10.5 Hz, J_{5,6}=6.3 Hz, 1H, H-6), 5.58 (d, J_{1,2}=1.8 Hz, 1H, H-1), 5.50 (ddd, J_{6,7cis}=17.3 Hz, J_{5,7cis=J}_{7cis,7trans}=1.3 Hz, 1H, H-7cis), 5.34 (ddd, J_{6,7}trans=10.5 Hz, J_{5,7trans}=J_{7cis,7trans}=1.3 Hz, 1H, H-7trans), 4.92 (AB, J_{AB}=10.6 Hz, 1H, CH₂Ph), 4.78 (AB, J_{AB}=12.4 Hz, 1H, CH₂Ph), 4.75-4.62 (m, 4H, CH₂Ph), 4.59 (dd, J_{4,5}=9.2 Hz, J_{5,6}=6.3 Hz, 1H, H-5), 4.05 (dd, J_{1,2}=1.8 Hz, J_{2,3}=2.8 Hz, 1H, H-2), 3.92 (dd, J_{2,3}=2.8 Hz, J_{3,4}=9.2 Hz, 1H, H-3), 3.86 (dd, J₃₄=J_{4,5}=9.2 Hz, 1H, H-4).

¹³C NMR (100 MHz, CDCl₃) δ (ppm) 138.3, 138.2, 137.8 (Cq, Ph), 134.9 (C-6), 134.4 (Cq, SPh), 131.5, 129.0, 128.4, 128.3, 128.1, 128.0, 127.9, 127.8, 127.8, 127.7, 127.4 (CH, Ph), 118.3 (C-7), 85.8 (C-1), 79.7 (C-3), 78.8 (C-4), 76.5 (C-2), 75.2 (CH₂Ph), 73.8 (C-5), 72.3, 72.1 (CH₂Ph).

### Step 4: Phenyl 2,3,4-tri-O-benzyl-1-thio-D-glycero-α-D-manno-heptopyranoside

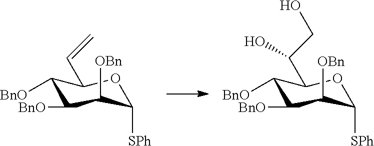

To a solution of K₂OsO₄ (0.20 g, 0.55 mmol), K₃Fe(CN)₆ (16.94 g, 51.46 mmol) and K₂CO₃ (7.92 g, 56.94 mmol) in a mixture of water (94 mL) and t-BuOH (94 mL) at 0° C. was added dropwise to a solution of alkene (9.9 g, 18.38 mmol) in toluene (37 mL) and the reaction was then allowed to warm to room temperature. After 48 h K₂OsO₄ (0.083 g, 0.23 mmol), K₃Fe(CN)₆ (2.54 g, 7.72 mmol) and K₂CO₃ (1.18 g, 8.49 mmol) were added and the mixture stirred for two more days. Then, Na₂SO₃ (32.2 g) was added, after 1.5 h, the reaction mixture was extracted with ethyl acetate (4×100 mL), the organic phases were washed with an aqueous solution of KOH 1M (100 mL), dried over MgSO₄, filtered and concentrated in vacuo. The residue was purified by silica gel column chromatography eluted with a gradient of cyclohexane/ethyl acetate (100/0 to 40/60) to afford the corresponding separated diols D and L with ratio 2/1 (total yield 7.74 g, 74%) as colorless oils with 51% yield for the D-isomer. Separated diols were assigned by transformation of D-stereoisomer into D-glycero-D-mannoheptose-7-phosphate substrate of HldE-kinase (Kosma et al. Carb. Res. 2005, 340, 2808), and transformation of the L-stereoisomer into ADP-L-heptose substrate of Waac and previously described in the literature (Kosma et al. Angew. Chem. Int. Ed. 2000, 39, 4150-4153).

[α]_{D}+103.3 (c1.00, CHCl₃).

MS (DCl—NH₃) m/z: 590 [M+NH₄]⁺

### Step 5: Thiophenyl 1-deoxy-2,3,4,6-tetra-O-benzyl-D-glycero-α-D-manno-heptopyranoside

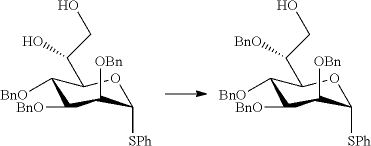

TIPSCI (3.36 mL, 15.85 mmol) was added dropwise at 0° C. to a solution of diol (6.05 g, 10.56 mmol) and imidazole (2.16 g, 31.69 mmol) in dry THF (47 mL). Then, the reaction was stirred at room temperature for 16 hours, the mixture was then concentrated, diluted with CH₂Cl₂ (290 mL), washed with saturated solution of ammonium chloride (2×160 mL), water (160 mL). The organic layer, dried over MgSO₄, filtered and the solvent removed by evaporation. Sodium hydride 60% in hexane (0.87 g, 21.84 mmol) was added to a solution of the previously obtained alcohol and dry DMF (80 mL). After 20 minute stirring under argon, benzylbromide (2.59 mL, 21.84 mmol) was added at 0° C. and the mixture stirred at room temperature for 3 h. The mixture was then diluted with Et₂O (520 mL) and successively washed with 1N HCl (173 mL), saturated solution of NaHCO₃ (173 mL) and water (173 mL). The aqueous phases extracted with diethyl ether (300 mL), organic phases dried over MgSO₄, filtered and the solvent removed by evaporation. After that, to a solution of the obtained crude in THF, TBAF was added and the mixture stirred for 17 hours at room temperature. The mixture was diluted with Et₂O (500 mL) and successively washed with saturated solution of ammonium chloride (173 mL×2), water (173 mL). The aqueous layers were extracted with Et₂O (500 mL). The organic layers dried over MgSO₄, filtered and the solvent removed by evaporation. The crude was purified on silica gel chromatography with a gradient of cyclohexane/ethyl acetate (100/0 to 60/40) to afford the corresponding alcohol (5.35 g, 76%).

MS-ESI (TOF-MS-ESI+): m/z: 685 [M+Na]⁺.

### Step 6: Thiophenyl 1-deoxy-2,3,4,6-tetra-O-benzyl-7-(dibenzyloxyphosphoryl)-D-glycero-α-D-manno-heptopyranoside

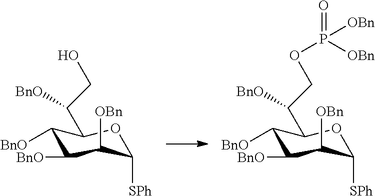

A solution of the previous alcohol (0.20 g, 0.3 mmol), PPh₃ (0.393 g, 1.5 mmol), dibenzylphosphate (0.417 g, 1.5 mmol) and triethylamine (417 mL, 3 mmol) in THF (2 mL) was prepared. Diethylazidocarboxylate 90% in toluene (235 μL, 1.5 mmol) was slowly added and the mixture was stirred for 24 h at room temperature. After concentration, the residue was purified by flash silica gel chromatography (cyclohexane/ethyl acetate, 4:1) to give the heptoside phosphate (0.25 g, 90%) as a colorless oil.

MS-ESI (TOF-MS-ESI+): m/z: 946 [M+Na]⁺.

### Step 7: 2,3,4,6-tetra-O-benzyl-7-(dibenzyloxyphosphoryl)-D-glycero-D-manno-heptopyranoside

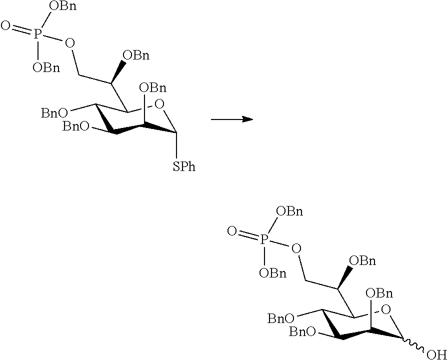

NBS (347 mg, 1.95 mmol) was added at 0° C. in absence of light to a solution of the thiophenyl derivative (900 mg, 975 μmol) in acetone (10 mL) and water (2 mL). After 4 hours, the mixture was quenched with saturated NaHCO₃, diluted with EtOAc, washed with saturated Na₂S₂O₄ and water. The aqueous phase were combined and extracted with EtOAc. The organic layer was dried with MgSO₄, filtered, concentrated and finally purified by flash chromatography (cyclohexane/EtOAc, 8:2→5:5) to yield the lactol intermediate (620 mg, 76%) as an oil.

MS (APCl+) m/z 853.3 [M+Na]⁺: 100%

### Step 8: Methyl 7-O-phosphoryl-D-glycero-α-D-manno-heptopyranoside

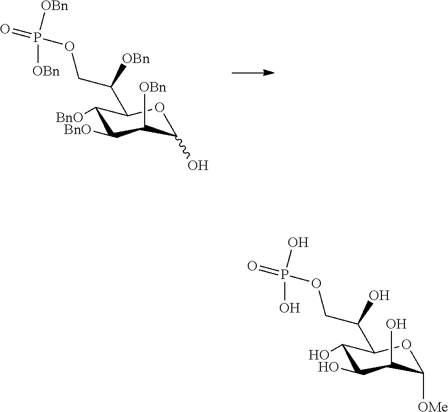

Iodomethane (86 μL, 570 μmol) was added dropwise to a solution of the previous lactol (158 mg, 190 μmol) and freshly prepared silver oxide Ag₂O (88 mg, 380 μmol) in 3 ml of dry DMF. The mixture was stirred at room temperature overnight under argon. The residue was diluted with EtOAc and filtered through celite. The filtrate was washed with saturated NH₄Cl and water. The organic layer was dried with MgSO₄, filtered, concentrated and finally purified by flash chromatography (cyclohexane/EtOAc, 7:3) to afford the methyl derivative (122 mg, 76%) as an oil. The anomeric configuration was ascertained after debenzylation by nuclear Overhauser effect NMR experiments and comparison with the β anomer.

MS (APCl+) m/z 845.3 [M+H]⁺: 100%

The previous derivative (52 mg, 61 μmol) was solubilised in a ternary solvent (EtOH/EtOAc/H₂O) and was hydrogenolyzed in the presence of Pd/C (10%, 85 mg) during two days. The residue was filtered through celite, washed with water and lyophilyzed to give the desired product (15 mg, 82%) as a white solid.

No through-space nOe correlations could be observed between H-1 and H-3 and/or H-1 and H-5, thus indicating an a anomeric configuration. Moreover, the corresponding β anomer was synthesized independently (see example 3 above). The proven β anomeric configuration for example 3 confirms the α configuration of example 2.

¹H NMR (D₂O, 400 MHz): δ (ppm) 4.75 (d, 1H, J₂₁=1.6 Hz, H1), 4.17-4.20 (m, 1H, H6), 4.05-4.10 (m, 1H, H7b), 3.92-3.96 (m, 1H, H7a), 3.90 (dd, 1H, J₂₁=1.6 Hz, J₂₃=3.4 Hz, H2), 3.81 (dd, 1H, J₄₅=9.8 Hz, J₄₃=9.5 Hz, H4), 3.72 (dd, 1H, J₃₄=9.5 Hz, J₃₂=3.4 Hz, H3), 3.69 (dd, 1H, J₅₄=9.8 Hz, J₅₆=2.7 Hz, H5), 3.41 (s, 3H, OMe).

¹³C NMR (D₂O, 100 MHz): δ (ppm) 100.9 (C₁), 72.6 (C5), 70.8 (C3), 70.7 (C6, J=6.7 Hz), 69.8 (C2), 67.2 (C4), 65.0 (C7), 54.8 (OCH₃). ³¹P NMR (D₂O, 101 MHz): δ (ppm) 2.79.

MS (ESI−) m/z 303.0 (M−H)⁻, 607.0 (2M-h)⁻.

HRMS (ESI+)C₈H₁₇O₁₃PNa measured 327.0462 calculated 327.0457.

### Example 3

### 1-O-Methyl-D-glycero-β-D-manno-heptopyranose 7-phosphate

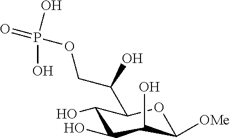

A suspension of known thiophenyl derivative (example 2, step 6, 200 mg, 0.22 mmol), NCS (58 mg, 0.44 mmol), molecular sieve 4 Å (130 mg) and MeOH dry (2.2 mL) was stirred at room temperature for one hour. Then, NCS (30 mg) and MeOH (1 mL) were added and the reaction mixture was stirred for 30 min. The reaction mixture was then concentrated under vacuum and directly purified by silica gel chromatography (elution with a mixture of cyclohexane/ethylacetate, 6/4) to afford 97 mg (52%) of alpha methyl and 70 mg (38%) of beta methyl anomers respectively.

MS (ESI+) m/z 867 [M+Na]⁺: 100%.

A mixture of the beta anomer (70 mg, 0.083 mmol) and Pd/C (10% 138 mg) in EtOH/EtOAc/H₂O (1.5 mL/0.9 mL/0.6 mL) was stirred under hydrogen atmosphere (1 bar) at room temperature for 24 hours. Then, the reaction mixture was filtered over celite, washed with water and lyophilized. The crude was purified by HPLC using a Zorbax C18-SB (Agilent) column (eluent H₂O, 81% yield).

MS (ESI+) m/z 327 [M+Na]. HRMS (ESI+) C₈H₁₇O₁₀PNa meas. 327.0462 cal c. 327.0457.

¹H NMR (400 MHz, D₂O) δ (ppm) 4.38 (bs, 1H, H-1), 4.02 (m, 1H, H-7), 3.89 (m, 1H, H-7), 3.81-3.75 (m, 2H, H-2 and H-7), 3.57 (t, H-4, 1H, J=9.4 and 9.8 Hz), 3.45 (dd, 1H, J=9.4 and 3.0 Hz, H-3), 3.37 (s, 3H, CH₃), 3.27 (dd, J=9.8 and 9.6 Hz, H-5).

¹³C NMR (100 MHz, D₂O) δ (ppm) 101.2 (C-1), 76.1 (C-5), 73.1 (C-3), 71.0 (d, C-6, J_{C-P}=6.7 Hz), 70.1 (C-2), 67.5 (C-4), 65.1 (C-7), 56.9 (CH₃).

³¹P NMR (D₂O, 101 MHz) δ 2.33

NOE 1D experiment showed through-space correlations between protons H1-H2, H1-H3 and H1-H5.

### Examples 4 and 5

### Ethyl 7-O-phosphoryl-D-glycero-α//β-D-manno-heptopyranoside

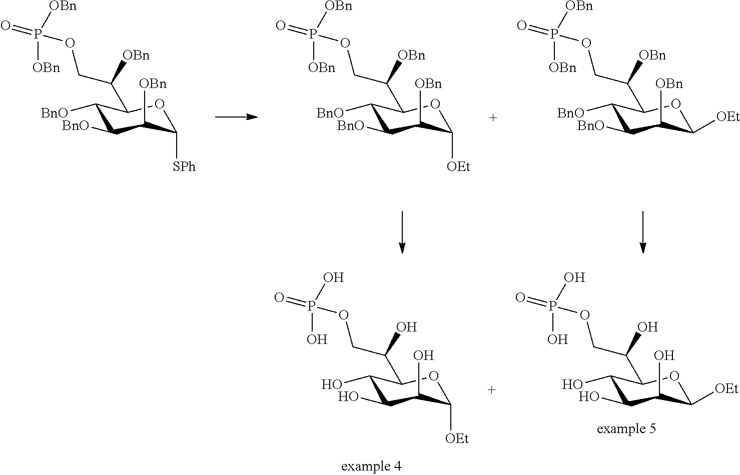

A suspension of known thiophenyl intermediate (example 2, step 6, 130 mg, 0.143 mmol), NCS (40 mg, 0.286 mmol), molecular sieves 4 Å (85 mg) and dry EtOH (2 mL) was stirred at room temperature for 6 hours. The reaction mixture was then concentrated under vacuum and directly purified by silica gel chromatography (elution with a mixture of cyclohexane/ethyl acetate,
6/4) to afford 40 mg (33%) of alpha ethyl and 30 mg (25%) of beta ethyl anomers, respectively.

MS of alpha and beta intermediates (ESI+) m/z 881 [M+Na]⁺: 100%.

### 1-O-Ethyl-D-glycero-α-D-manno-heptopyranose 7-phosphate (example 4)

A suspension the corresponding compound (40 mg, 47 μmol), and Pd/10% C (80 mg) in EtOH/EtOAc/H₂O (1.5, 0.9, 0.6) mL was stirred under hydrogen atmosphere (1 bar) at room temperature for 24 hours. Then, the reaction mixture was filtered over celite, washed with water and lyophilized. The crude was purified using a HPLC using a Zorbax C18-SB (Agilent) column (eluent H₂O) to yield 7 mg (50% yield) of the a ethoxy analogue.

HRMS (ESI+)C₉H₁₉O₁₀PNa meas. 341.0600 calc. 341.0614.

¹H NMR (400, D₂O, 25° C.) δ 4.73 (s, 1H, H-1), 3.71-3.74 (m, 2H, H-6 and H-7), 3.64-3.67 (m, 3H, H-7, H-2 and H-4), 3.59-3.62 (m, 3H, CH, H-2 and H-5), 3.38-3.42 (m, 1H, CH), 1.08 (t, 1H, CH₃, J=7.0 Hz).

¹³C NMR (100 MHz, D₂O) δ 99.4 (C-1), 72.8 (C-5), 70.9 (C-3), 70.6 (d, C-7, J=6.5 Hz), 70.0 (C-2), 67.4 (C-4), 66.5 (d, C-6, J=4.8 Hz), 63.5 (CH₂), 14.0 (CH₃).

³¹P NMR (D₂O, 101 MHz) δ (ppm) 1.04.

### 1-Ethyl-D-glycero-β-D-manno-heptopyranose 7-phosphate (example 5)

A suspension the corresponding compound (30 mg, 35 μmol), and Pd/10% C (60 mg) in EtOH/EtOAc/H₂O (1.5, 0.9, 0.6) mL was stirred under hydrogen atmosphere (1 bar) at room temperature for 24 hours. Then, the reaction mixture was filtered over celite, washed with water and lyophilized. The crude was purified using a HPLC using a Zorbax C18-SB (Agilent) column (eluent H₂O) to yield 3 mg (30% yield) of the desired β ethoxy analogue.

HRMS (ESI+)C₉H₁₉O₁₀PNa measured 341.0615 calculated 341.0614.

¹H NMR (400 MHz, D₂O) β (ppm) 4.51 (s, 1H, H-1), 4.08-4.02 (m, 1H, H-6), 3.98-3.91 (m, 1H, H-7), 3.86-3.76 (m, 3H, H-2, CH, H-7b), 3.62-3.58 (m, 2H, H-4 and CH), 3.46 (dd, 1H, J=8.0 and 4.0 H, H-3), 3.28 (dd, 1H, H-5, J=12.0 and 3.4 Hz), 1.08 (t, 3H, CH₃, J=7.0 Hz).

¹³C NMR (100 MHz, D₂O) δ (ppm) 99.8 (C-1), 75.8 (C-5), 73.2 (C-3), 71.0 (d, C-7, J=6.5 Hz), 70.4 (C-2), 67.6 (C-4), 65.6 (C-6), 63.5 (CH₂), 14.2 (CH₃).

³¹P NMR (D₂O, 101 MHz) δ (ppm) 1.23.

NOE 1D experiment showed through-space correlations between protons H1-H2, H1-H3, H1-H4 and H1-H5.

### Example 6

### α-Fluoro 7-O-phosphoryl-D-glycero-α-D-manno-heptopyranoside

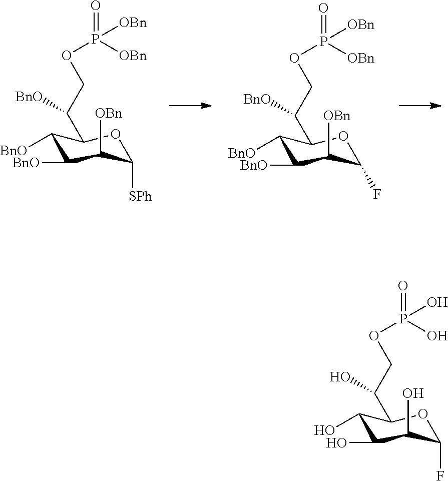

NBS (169 mg, 905 μmol) was added at 0° C. in absence of light to a solution of know thiophenyl derivative (example 2, step 6, 438 mg, 474 μmol) in acetone (10 mL) and water (2 mL). After 4 hours, the mixture was quenched with saturated NaHCO₃, diluted with EtOAc, washed with saturated Na₂S₂O₄ and water. The aqueous phase were combined and extracted with EtOAc. The organic layer was dried with MgSO₄, filtered, concentrated and finally purified by flash chromatography (cyclohexane/EtOAc, 8:2→5:5) to yield the lactol intermediate (302 mg, 76%) as an oil.

MS (APCI+) m/z 853.3 [M+Na]⁺.

HRMS calculated for C₄₉H₅₁O₁₀PNa [M+Na]⁺: 853.3094. found: 853.3112.

To a solution of lactol intermediate (302 mg, 363 μmol) in dry THF (10 mL) at −30° C., was added DAST (134 μL, 1.09 mmol) dropwise under argon. The reaction mixture was allowed to warm to room temperature during 7 hours. Then H₂O (50 mL) was added. The aqueous layer was extracted with ethyl acetate (20 mL, 3 times). The combined organic extracts were washed with brine (20 mL) and dried (MgSO₄). Solvent evaporation and flash chromatography on silica gel (8:2 pentane/EtOAc) afforded 253 mg (83%) of α-fluoro intermediate as an oil.

MS (ESI+) m/z 855.3 [M+Na]⁺, HRMS calculated for C₄₉H₅₀FO₉PNa [M+Na]⁺: 855.3016. found: 855.3069; and 31 mg (10%) of β-fluoro intermediate as an oil, MS (ESI+) m/z 855.3 [M+Na]⁺, HRMS calculated for C₄₉H₅₀FO₉PNa [M+Na]⁺: 855.3027. found: 855.3069.

The alpha anomer (94 mg, 114 μmol) was solubilised in a binary solvant (THF/MeOH: 2 mL/4 mL) and was hydrogenolised in the presence of Pd/C (10%, 95 mg) during two days according to previously described procedures. The residue was filtered through celite, washed with water and lyophilised to give the desired product (24 mg, 72%) as a white solid.

¹H NMR (D₂O, 400 MHz): δ (ppm) 5.61 (d, 1H, J_{HF}=49.2 Hz, H1), 4.17-4.20 (m, 1H, H6), 4.06 (m, 1H, H2), 3.97-4.03 (m, 1H, H7a), 3.84-3.93 (m, 3H, H4, H5, H7b), 3.78-3.81 (m, 1H, H3).

³¹P NMR (D₂O, 101 MHz): δ (ppm) 4.60.

¹⁹F NMR (D₂O, 400 MHz): δ (ppm)-139.17 (d, J_{HF}=49.2 Hz).

MS (ESI−) m/z 303.0 (M−H)⁻, 607.0 (2M-h)⁻.

HRMS (ESI+) C₇H₁₄O₉FPNa meas. 315.0263 calc. 315.0257.

NOE 1D experiment showed through-space absence of correlations with protons H5 or H3.

### Example 7

### 7-O-phosphoryl-1-deoxy-D-glycero-D-manno-heptopyranoside

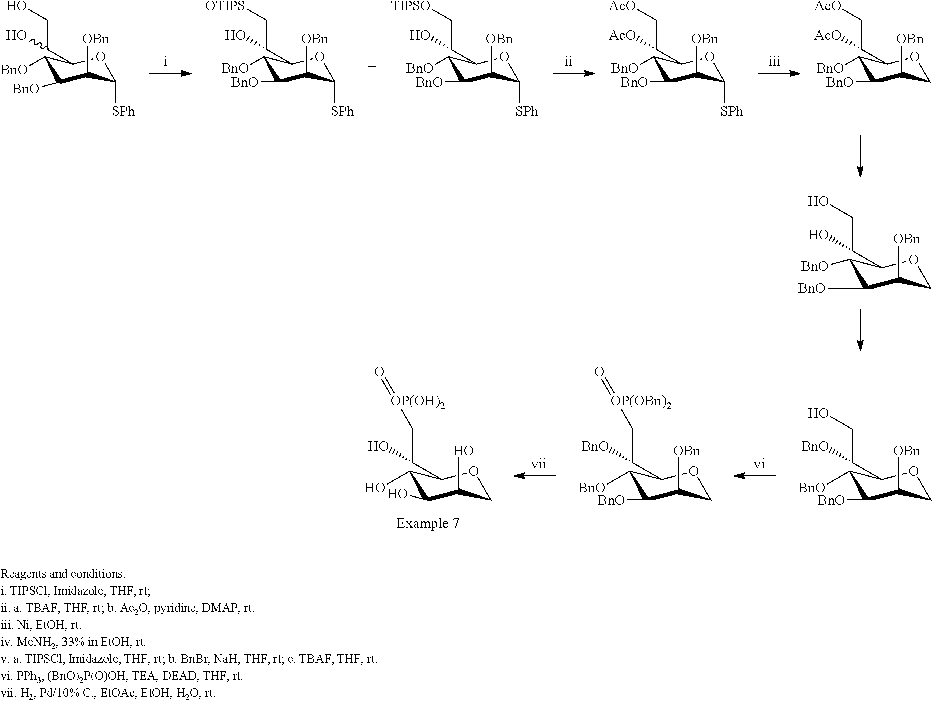

### Step 1: 1-Deoxy-1-thiophenyl-2,3,4-tri-O-benzyl-7-triisopropylsilyl-D-glycero-α-D-manno-heptopyranose

TIPSCI (0.55 mL, 2.62 mmol) was added dropwise at 0° C. to a solution of D/L diol (example 2, step 4) (1 g, 1.75 mmol) and imidazole (0.36 g, 5.23 mmol) in dry THF (8 mL). Then, the reaction was stirred at room temperature for 16 hours, the mixture was then concentrated, diluted with CH₂Cl₂ (50 mL), washed with saturated solution of ammonium chloride (2×30 mL) and water (30 mL). The organic layer, dried over MgSO₄, filtered and the solvent removed by evaporation. The residue was purified with a silica gel column chromatography eluted with a gradient of cyclohexane/EtOAc (100/0 to 40/60), yielding 800 mg of the desired D-stereoisomer (70%), which was assigned by transformation of D-stereoisomer into D-glycero-D-mannoheptose-7-phosphate substrate of HldE-kinase (Kosma et al. Carb. Res. 2005, 340, 2808).

MS (ESI+) m/z 752.05 [M+Na]⁺.

### Step 2: 1-Deoxy-1-thiophenyl-6,7-di-O-acetyl-2,3,4-tri-O-benzyl-D-glycero-α-D-manno-heptopyranose

A solution of the previous compound (1.9 g, 2.606 mmol), THF (25 mL) and TBAF (3.30 g, 10.424 mmol) was stirred for 17 hours at room temperature. The mixture was diluted with Et₂O (100 mL) and successively washed with saturated solution of ammonium chloride (50 mL×2) and water (50 mL). The aqueous layers, extracted with Et₂O (100 mL). The organic layers dried over MgSO₄, filtered and the solvent removed by evaporation. The crude was purified on silica gel chromatography with a gradient of cyclohexane/ethyl acetate (100/0 to 60/40) to afford the corresponding alcohol. The latter, and 4-DMAP (0.03 g, 0.262 mml) were dissolved in dry pyridine (30 mL), and acetic anhydride added dropwise. Then, the mixture stirred at room temperature overnight. After that, (50 mL) of brine were added, the mixture extracted with EtOAc (3×50 mL), the organic layers dried, filtered and solvent removed by evaporation. The crude purified with a silica gel column chromatography eluted with a mixture cyclohexane/EtOAc (40/60), yielding 1.13 g of product (70% yield).

MS (ESI+) m/z 679.24 [M+Na]⁺.

### Step 3: 1-Deoxy-2,3,4-tri-O-benzyl-6,7-di-O-acetyl-D-glycero-D-manno-heptopyranose

A suspension of the previous intermediate (0.273 g, 0.361 mmol), Raney Nickel (1.5 g) and ethanol was stirred for 4 hours at room temperature. Then, the same amount of Raney Nickel was added once more and the mixture was stirred for 2 hours at the same temperature. Then, the suspension was filtered over celite, and the solvent removed under vacuum, yielding 150 mg of desired product (76% yield).

MS (ESI+) m/z 571.23 [M+Na]⁺: 100%.

### Step 4: 1-Deoxy-2,3,4-tri-O-benzyl-D-glycero-D-manno-heptopyranose

The previous intermediate (0.15 g, 0.274 mmol) was dissolved in 33% solution of methylamine in ethanol (6 mL) and stirred at room temperature overnight. Then, the solvent was removed under vacuum and the crude reaction mixture was purified by silica gel column chromatography eluted with a 6/4 solution of cyclohexane and ethyl acetate providing 90 mg of desired product (71% yield).

MS (ESI+) m/z 487.0 [M+Na]⁺: 100%.

### Step 5: 1-Deoxy-2,3,4,6-tetra-O-benzyl-D-glycero-D-manno-heptopyranose

TIPSCI (0.06 mL, 0.29 mmol) was added dropwise at 0° C. to a solution of previous compound (90 mg, 0.194 mmol), imidazole (40 mg, 0.581 mmol) in dry THF (5 mL). After 16 hours at room temperature, the mixture was concentrated, diluted with dichloromethane (10 mL), washed with ammonium chloride (10 mL×2), then water (10 mL). The organic layer was dried, filtered, and the solvents were removed under vacuum. The resulting mixture was directly dissolved in dry DMF (2 mL). Then, NaH (60%, 15 mg, 0.388 mmol) was added to this solution. After 20 min, BnBr (46 μL, 0.388 mmols) was added at 0° C. and the mixture was stirred at room temperature for 3 hours. The mixture was diluted with Et₂O (20 mL) and successively washed with 1N HCl (5 mL), saturated NaHCO₃ (5 mL) and water (5 mL). The aqueous layers were extracted once more with Et₂O (30 mL), the organic layers were dried, filtered and the solvent was removed under vacuum. Finally, the crude was dissolved in dry THF (5 mL) and TBAF (245 mg, 0.776 mmol) was added. The mixture was stirred at room temperature for 17 hours, then diluted with Et₂O (10 mL) and successively washed with saturated ammonium chloride (5 mL) and water (5 mL). The aqueous layers were extracted once more with Et₂O (30 mL). The combined organic layers were dried, filtered and the solvent was removed under vacuum. The residual crude was purified using silica gel chromatography (ethyl acetate/cyclohexane, 4/6) yielding 45 mg of product (42%).

MS (ESI+) m/z 577.27 [M+Na]⁺: 100%.

### Step 6: 1-Deoxy-2,3,4,6-tetra-O-benzyl-7-O-dibenzyloxyphosphoryl-D-glycero-D-manno-heptopyranose

To a solution of the previous alcohol (32 mg, 0.061 mmol), PPh₃ (80 mg, 0.305 mmol), (Bn)₂P(O)OH (85 mg, 0.305 mmol), TEA (85 μL, 0.609 mmol) and THF (2 mL), was slowly added DEAD (48 μL, 0.305 mmol) at room temperature and the mixture was stirred overnight. Then, the solvent was removed and the crude purified by silica gel chromatography (elution with ethyl acetate/cyclohexane, 4/6) to give the desired compound (40 mg, 82% yield).

MS (ESI+) m/z 846.32 [M+Na]⁺: 100%.

### Step 7: 1-Deoxy-D-glycero-D-manno-heptopyranose 7-phosphate

A suspension of the previous heptoside (40 mg, 0.049 mmol), and Pd/C (10%, 90 mg) in EtOH/EtOAc/H₂O (1.5, 0.9, 0.6) mL was stirred under hydrogen atmosphere (1 bar) at room temperature for 24 hours. Then, the reaction mixture filtered over celite, washed with water and lyophilized. The crude purified by HPLC (Zorbax SB-C18) with a water isocratic system of elution yielding 5 mg of the desired product (38%).

HRMS (ESI+) C₇H₁₅O₉PNa measured 297.0345 calculated 297.0351.

MS (ESI+) m/z 297 [M+Na]⁺: 100%.

¹H NMR (400 MHz, D₂O) δ ppm 4.04-4.02 (m, 2H, H-6 and H-7b), 3.73-3.78 (m, 3H, H-1b, H-4, and H-7a), 3.57 (t, 1H, H-4, J=7.8 and 9.8 Hz), 3.42-3.45 (m, 2H, H-1a and H-3), 3.20 (d, 1H, H-5, J=9.2 Hz).

¹³C NMR (100 MHz, D₂O) δ ppm 80.8 (C-5), 73.8 (C-3), 70.5 (C-6, d, J=7.7 Hz), 70.2 (C-1), 68.9 (C-2), 67.5 (C-4), 65.4 (d, C-7, J=4.8 Hz).

³¹P NMR (D₂O, 101 MHz) δ ppm 1.40.

### Example 8

### 1-Deoxy-1-propyl-D-glycero-α-D-manno-heptopyranose 7-phosphate

### Example 9

### 1-Deoxy-1-propyl-D-glycero-β-D-manno-heptopyranose 7-phosphate

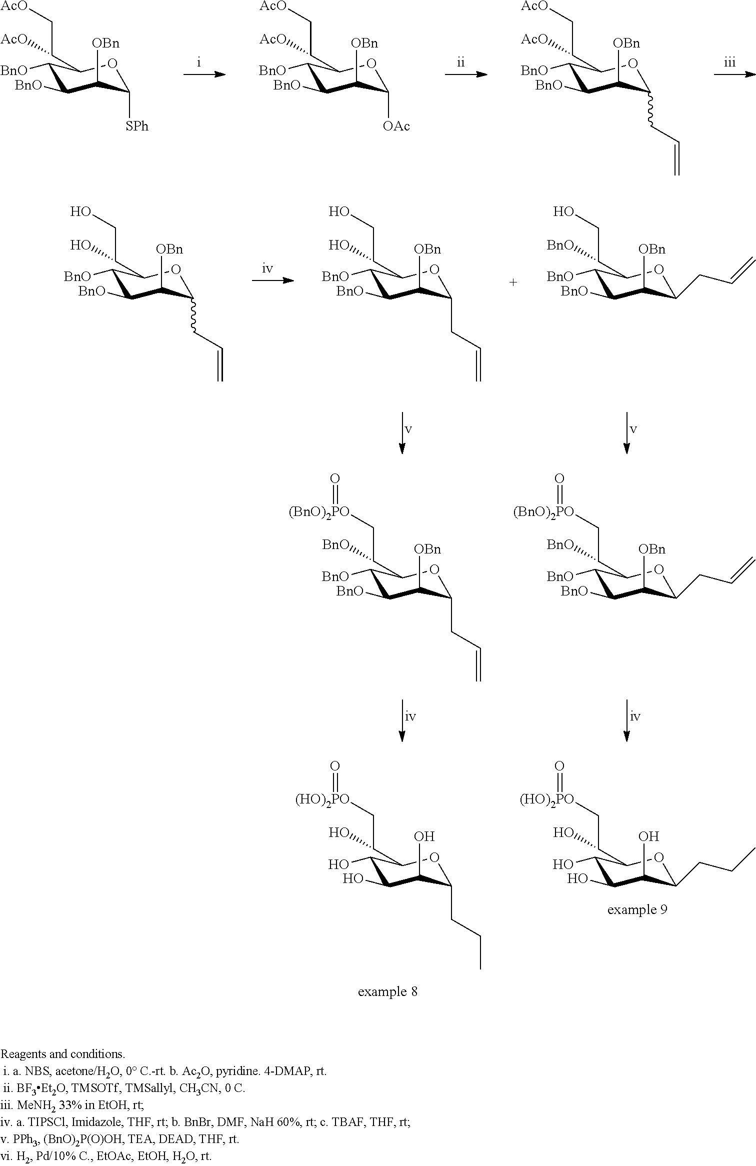

### Step 1: 1,6,7-tri-O-acetyl-2,3,4-tri-O-benzyl-D-glycero-α-D-manno-heptopyranoside

NBS (0.25 g, 1.40 mmol) was added at −15° C. in absence of light to a solution of known phenyl 6,7-di-O-acetyl-2,3,4-tri-O-benzyl-1-thio-D-glycero-α-D-manno-heptopyranoside (example 7, step 2) (0.46 mg, 0.70 mmol) in acetonitrile (18 mL). After 6 hours, the mixture was quenched with saturated NaHCO₃, diluted with EtOAc, washed with saturated Na₂S₂O₄ and water. The aqueous phases were combined and extracted with EtOAc. The organic layer was dried with MgSO₄, filtered, and concentrated. The crude directly dissolved in dry pyridine (2 mL), the 4-DMAP was added (0.008 g, 0.070 mmol) and finally the anhydride acetic added dropwise and the mixture stirred for 6 hours at room temperature. After that, (50 mL) of brine were added, the mixture extracted with EtOAc (3×50 mL), the organic layers dried, filtered and solvent removed by evaporation. The crude was purified with a silica gel column chromatography eluted with a mixture cyclohexane/EtOAc (40/60) to afford the desired product (0.38 g, 90% yield).

MS (ESI+) m/z 629.24 [M+Na]⁺.

### Step 2: 1-Deoxy-1-allyl-6,7-di-O-acetyl-2,3,4-tri-O-benzyl-D-glycero-α,β-D-manno-heptopyranose

To a solution of previous heptopyranoside (1.6 g, 2.64 mmol), trimethylallylsilane (0.85 mL, 5.28 mmol), and CH₃CN (20 mL), was added BF₃.Et₂O (1.1 mL, 7.92 mmol) dropwise at 0° C. Then, trimethylsilyltriflate (1.6 mL, 7.92 mmol) was added at the same temperature and the reaction stirred 3 hours. After that, a saturated solution of NaHCO₃ was introduced at 0° C. until the mixture was neutralized (pH=7), then the solution was extracted with ethyl acetate (100 mL×3). The organic layers were dried, filtered and the solvent removed under vacuum. The obtained crude was purified by silica gel chromatography, (elution with ethyl acetate/cyclohexane, 40/60) to give 810 mg of the compound as a mixture of α/β isomers.

MS (ESI+) m/z 611.2 [M+Na]⁺100%.

The α/β assignment was based on literature data: all the C-allylation reported in the literature of mannosides protected by benzyl or acetate groups always give the α anomer as the major stereoisomers, without exception. The α/β ratios are in the range of 2/1 and 3/1 for the least selective methods (Carbohydr. Res. 341 (2006) 1708-1716, Org. Lett. 10 (2008) 4731-4734). The other methods only describe the α anomer or α/β selectivities up to 15/1 (J. Am. Chem. Soc. 104 (1982) 4976-4978, Carbohydr. Res. 223 (1992) 243-253, Tetrahedron Lett. 25 (1984) 2383-2386, Carbohydr. Res. 171 (1987) 223-232, Org. Lett. 3 (2001) 1547-1550, J. Am. Chem. Soc. 123 (2001) 9545-9554).

### Step 3: 1-Deoxy-1-allyl-2,3,4-tri-O-benzyl-D-glycero-α,β-D-manno-heptopyranose

The previous compound (200 mg, 340 μmol) was treated with 33% methylamine in ethanol (7 mL) and stirred at room temperature overnight. 50 mL of water was added and the mixture extracted with ethyl acetate (50 mL×3). The organic layers were dried, filtered and the solvent was removed under vacuum. The obtained crude was purified using silica gel chromatography (elution with ethyl acetate/cyclohexane, 50/50) to give 163 mg (95%, yield) of the desired diol as a mixture of major and minor compounds. The NMR spectra attribution was based on literature data describing that such an allylation on mannosides always give the α anomer as the major stereoisomer (see justifications, above). Two sets of peaks are present in both ¹H and ¹³C spectra. A definitive proof of the α/β structure could not be provided by noesy experiments at this stage but the two anomers have been separated in the next step.

¹H NMR (400, CDCl₃, 25° C.) δ 7.42-7.40 (m, 18H, 18H_{arom, maj}), 7.38-7.36 (m, 18H, 18H_{arm, min}), 5.71-5.68 (m, 1H, H_{b, maj})_{,} 5.68-5.65 (m, 1H, H_{b, min}), 5.08-5.04 (m, 2H, H_{a,a′, maj}), 5.08-5.04 (m, 2H, H_{a,a′, min}), 4.84 (d, J_{H/H}=10.8 Hz, H_{arom, min}), 4.78 (d, J_{H/H}=11.5 Hz, H_{arom,maj}), 4.63-4.57 (m, 5H, H_{arom min}), 4.63-4.57 (m, 5H, H_{arom maj}), 4.07-4.03 (m, 3H, H-1 ₘᵢₙ, H-4ₘᵢₙ, and H-6ₘᵢₙ), 4.01-3.96 (m, 3H, H-1ₘₐⱼ, H-4ₘₐⱼ and H-6ₘₐⱼ), 3.82 (dd, 1H, J_{H2/H3} and J_{3/H4}=6.9 and 6.9 Hz, H-3ₘₐⱼ), 3.78 (dd, 1H, J_{4/5} and J_{5/6}=8.5 and 11.5 Hz, H-5ₘᵢₙ), 3.70-3.65 (m, 4H, H-2ₘₐⱼ, H-5_{α}, 2×H-7ₘₐⱼ), 3.68-3.64 (m, 3H, H-2ₘᵢₙ and 2×H-7ₘᵢₙ), 3.55 (dd, J_{H2/3,3/4}=8.2 and 8.1 Hz, 1H, H-3ₘᵢₙ), 2.34 (m, 2H, H_{c,c′maj}), 2.16 (m, 2H, H_{c,c′min}).

MS (ESI+) m/z 527 [M+Na]⁺: 100%.

### Step 4: 1-Deoxy-1-allyl-2,3,4,6-tetra-O-benzyl-D-glycero-α-D-man no-heptopyranose and 1-Deoxy-1-allyl-2,3,4,6-tetra-O-benzyl-D-glycero-β-D-man no-heptopyranose

TIPSCI (0.17 mL, 0.803 mmol) was added dropwise at T=0° C. to a solution of previous diol (270 mg, 0.535 mmol), imidazole (110 mg, 1.606 mmol) and dry THF. After 16 hours at room temperature, the mixture was concentrated, diluted with dichloromethane (50 mL), washed with ammonium chloride (20 mL×2), then water (20 mL). The organic layer was dried, filtered, and the solvent was removed under vacuum. The obtained crude was directly dissolved in dry DMF (4 mL). Then, NaH (60%, 43 mg, 1.07 mmol) was added to this solution. After 20 min, BnBr (130 mg, 1.07 mmols) was added at T=0° C. and the mixture was stirred at room temperature for 3 hours. The mixture was diluted with Et₂O (30 mL) and successively washed with 1N HCl (10 mL), sat. NaHCO₃ (10 mL) and water (10 mL). The aqueous layers were extracted once more with Et₂O (30 mL), the organic layers were dried, filtered and the solvent was removed under vacuum. Finally, the crude was dissolved in dry THF and TBAF was added. The mixture was stirred at room temperature for 17 hours, then diluted with Et₂O (30 mL) and successively washed with sat. ammonium chloride (10 mL) and water (10 mL). The aqueous layers were extracted once more with Et₂O (30 mL). The combined organic layers were dried, filtered and the solvent was removed under vacuum. The residual crude was purified using silica gel chromatography (gradual elution from 0 to 40% of ethyl acetate/cyclohexane), to provide the desired α- (70 mg) and β-anomers (50 mg) (see justifications for anomeric assignment above).

MS (ESI+) m/z 617[M+Na]⁺: 100%.

### Step 5: 1-Deoxy-1-allyl-2,3,4,6-tetra-O-benzyl-7-O-dibenzyloxyphosphoryl-D-glycero-α-D-manno-heptopyranose and 1-Deoxy-1-allyl-2,3,4,6-tetra-O-benzyl-7-dibenzyloxyphosphoryl-D-glycero-β-D-manno-heptopyranose

To a solution of previous α or β-anomers (0.082 mmol), PPh₃ (0.421 mmol), (BnO)₂P(O)OH (0.402 mmol), TEA (0.842 mmol) and THF (2 mL), was slowly added DEAD (0.421 mmol) at room temperature and the mixture was stirred for 4 days. Then, the solvent was removed and the residual crude purified by silica gel chromatography (elution with ethyl acetate/cyclohexane, 40/60) to provide the desired compounds.

1-Deoxy-1-allyl-2,3,4,6-tetra-O-benzyl-7-O-dibenzyloxyphosphoryl-D-glycero-α-D-manno-heptopyranose (70 mg, 98 yield).

MS (ESI+) m/z 877 [M+Na]⁺: 100%.

### 1-Deoxy-1-allyl-2,3,4,6-tetra-O-benzyl-7-dibenzyloxyphosphoryl-D-glycero-β-D-manno-heptopyranose (30 mg, 70% yield).

MS (ESI+) m/z 877 [M+Na]⁺: 100%.

### Step 6: 1-Deoxy-1-propyl-D-glycero-α-D-manno-heptopyranose 7-phosphate and 1-Deoxy-1-propyl-D-glycero-β-D-manno-heptopyranose 7-phosphate

### 1-Deoxy-1-propyl-D-glycero-α-D-manno-heptopyranose 7-phosphate:

A suspension of the previous α-anomer (70 mg, 0.082 mmol), and Pd/C (10%, 202 mg) in EtOH/EtOAc/H₂O (1.5, 0.9, 0.6) mL was stirred under hydrogen atmosphere (1 bar) at room temperature for 48 hours. Then, the reaction mixture filtered over celite, washed with water and lyophilized. The crude was purified by HPLC (Zorbax SB-C18) with a water isocratic system of elution yielding 9 mg of the desired compound (37% yield).

HRMS for C₁₀H₂₁O₉PNa Meas. 339.0821 calc. 339.0821

NMR proton attribution determined by COSY.

¹H NMR (400, D₂O, 25° C.) δ 4.04-4.06 (m, 1H, H-1) 3.90-3.98 (m, 1H, H-6), 3.40-3.79 (m, 5H, H-2, H-4, H-3, and 2H-7), 3.46-3.53 (m, 1H, H-5), 1.57-1.64 (m, 1H, CH₂), 1.30-1.37 (m, 2H, CH₂), 1.12-120 (m, 1H, CH₂),0.80 (t, 3H, CH₃, J=6.9 Hz).

³C NMR (100 Hz, D₂O, 25° C.) δ 77.7 (C-1), 73.5 (C-2), 71.1 (C-5), 71.0 (d, C-6, J=6.5 Hz), 67.8 (C-3), 65.8 (C-4), 66.5 (d, C-7, J=4.8 Hz), 29.8 (CH₂), 18.5 (CH₂), 13.0 (CH₃).

³¹P NMR (D₂O, 101 MHz) δ 1.24.

### 1-Deoxy-1-propyl-D-glycero-β-D-manno-heptopyranose 7-phosphate:

A suspension of the corresponding compound (30 mg, 41 μmol), and Pd/10% C (100 mg) in EtOH/EtOAc/H₂O (1.5, 0.9, 0.6) mL was stirred under hydrogen atmosphere (1 bar) at room temperature for 48 hours. Then, the reaction mixture filtered over celite, washed with water and lyophilized. The crude purified by HPLC (Zorbax SB-C18) with water as eluant yielding 4 mg (31% yield) of the desired product.

HRMS for C₁₀H₂₁O₉PNa Meas. 339.0829 calc. 339.0821 NMR proton attribution determined by COSY.

¹H NMR (400, D₂O, 25° C.) δ 4.02 (t, 1H, H-6, J=5.0 Hz), 3.87-3.95 (m, 3H, 2× H-7 and H-1), 3.70-3.85 (m, 3H, H-2, H-4 and H-3), 3.38 (d, 1H, H-5, J=7.6 Hz), 1.64 (m, 1H, CH₂), 1.30 (m, 2H, CH₂), 1.15 (m, 1H, CH₂), 0.78 (t, 1H, CH₃, J=7.0 Hz).

¹³C NMR (100 Hz, D₂O, 25° C.) δ 78.3 (C-1), 71.8 (C-2), 71.7 (C-5), 71.2 (C-3), 67.9 (d, C-6, J=6.5 Hz), 66.7 (C-4), 66.5 (d, C-7, J=4.8 Hz), 29.4 (CH₂), 18.6 (CH₂), 13.0 (CH₃).

³¹P NMR (D₂O, 101 MHz) δ 1.04.

### Example 10

### 1-Deoxy-D-glycero-D-manno-heptopyranose

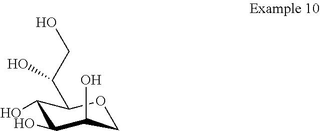

### Step 1: 1-Deoxy-2,3,4-tri-O-benzyl-D-glycero-D-manno-heptopyranose

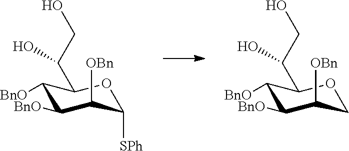

Raney-nickel (3 g) was washed with absolute EtOH (3×15 ml) and added as a suspension in absolute EtOH (30 ml) to phenyl 2,3,4-tri-O-benzyl-1-thio-D-glycero-α-D-manno-heptopyranoside (see example 2, step 4, 150 mg, 0.262 mmol). The suspension was stirred at room temperature under argon atmosphere for 3 h. The mixture was filtered over celite, and the residue was washed with absolute EtOH (5×10 ml). The organic layer was concentrated and finally purified by flash chromatography (cyclohexane/EtOAc, 7:3) to yield the desired compound (93 mg, 76%) as a white solid.

¹H NMR (CDCl₃, 400 MHz): δ (ppm): 7.28-7.39 (m, 15H, H^{arom}), 5.08 (AB, J_{AB}=10.8 Hz, 1H, CH₂^{Bn}), 4.74 (AB, J_{AB}=12.6 Hz, 1H, CH₂^{Bn}), 4.67-4.69 (m, 2H, CH₂^{Bn}), 4.64 (AB, J_{AB}=11.7 Hz, 1H, CH₂^{Bn}), 4.55 (AB, J_{AB}=11.7 Hz, 1H, CH₂^{Bn}), 4.06 (dd, J_{1b-2}=2.2 Hz, J_{1b-1a}=12.6 Hz, 1H, H-1b), 3.98 (dd, J₄₋₃=9.2 Hz, J₄₋₅=9.4 Hz, 1H, H-4), 3.90 (m, 1H, H-6), 3.76-3.78 (m, 2H, H-2, H-7b), 3.63 (dd, J₃₋₂=3.2 Hz, J₃₋₄=9.2 Hz, 1H, H-3), 3.61 (m, 1H, H-7a), 3.37 (dd, J₅₋₆=4.6 Hz, J₅₋₄=9.4 Hz, 1H, H-5), 3.27 (d, J_{1a-1b=}12.6 Hz, 1H, H-1a), 3.25 (s, 1H, OH), 2.22 (s, 1H, OH). ¹³C NMR (101 MHz, CDCl₃, 25° C.) δ (ppm): 138.0 (Cq^{arom}), 137.9 (Cq^{arom}), 137.8 (Cq^{arom}), 128.5 (CH^{arom}), 128.4 (CH^{arom}), 128.1 (CH^{arom}), 127.8 (CH^{arom}), 127.7 (CH^{arom}), 127.6 (CH^{arom}), 83.0 (C-3), 80.5 (C-5), 76.2 (C-4), 75.1 (CH₂^{Bn}), 72.3 (C-2), 72.0 (C-6), 71.4 (CH₂^{Bn}), 71.2 (CH₂^{Bn}), 67.0 (C-1), 62.9 (C-7).

MS (APCI+) m/z: 465.2 [M+Na]⁺.

### Step 2: 1-Deoxy-D-glycero-D-manno-heptopyranose

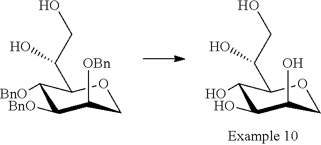

The previous intermediate (70 mg, 0.151 μmol) was solubilised in a ternary solvant system (MeOH/THF/H₂O, 3 mL/2 mL/2 mL) and was hydrogenolized in the presence of Pd/C (10%, 70 mg) during two days. The residue was filtered through celite, washed with water and lyophilised to give the desired product (23 mg, 80%) as a white solid;

¹H NMR (D₂O, 400 MHz): δ (ppm): 3.99 (ddd, J₆₋₅=3.1 Hz, J_{6-7b}=3.4 Hz, J₆₋₇ₐ=7.6 Hz, 1H, H-6), 3.93 (m, 1H, H-2), 3.88 (dd, J_{1b-2}=1.9 Hz, J_{1b}-1a=12.7 Hz, 1H, H-1b), 3.75 (dd, J_{7b-6}=3.4 Hz, J_{7b-7a}=12.0 Hz, 1H, H-7b), 3.67 (dd, J₄₋₃=9.4 Hz, J₄₋₅=9.7 Hz, 1H, H-4), 3.66 (dd, J₇ₐ₋₆=7.6 Hz, J_{7a-7b}=12.0 Hz, 1H, H-7a), 3.59 (dd, J₃₋₂=3.4 Hz, J₃₋₄=9.4 Hz, 1H, H-3), 3.56 (dd, J₁ₐ₋₂=0.7 Hz, J_{1a-1b}=12.7 Hz, 1H, H-1a), 3.31 (dd, J₅₋₆=3.1 Hz, J₅₋₄=9.7 Hz, 1H, H-5). ¹³C NMR (101 MHz, D₂O, 25° C.) δ (ppm): 81.1 (C5), 73.8 (C3), 71.7 (C6), 70.2 (C1), 68.9 (C2), 67.8 (C4), 61.6 (C7).

MS (ESI+) m/z 217.0 [M+Na]⁺; HRMS calcd. for C₇H₁₄O₉Na [M+Na]⁺: 217.0683. found: 217.0672.

### Example 11

### 1-β-C-Hydroxymethylene 1-Deoxy-7-O-phosphoryl-D-glycero-D-manno-heptopyranose

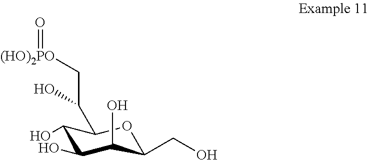

### Step 1: 2,3,4,6-tetra-O-benzyl-7-(dibenzyloxyphosphoryl)-D-glycero-D-manno-hepto-1,5-pyranone

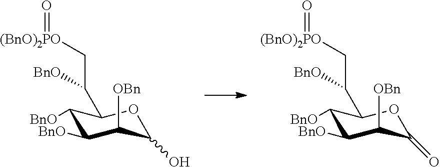

A mixture of 2,3,4,6-tetra-O-benzyl-7-(dibenzyloxyphosphoryl)-D-glycero-D-manno-heptopyranoside (Example 2, step 7, 100 mg, 0.12 mmol), molecular sieve 4 Å (180 mg) and anhydrous dichloromethane (2 mL) was stirred at room temperature for 30 min. Then, pyridinium chlorochromate (100 mg, 0.48 mmol) was added and the reaction mixture was stirred 4 hours at the same temperature. A mixture of cyclohexane/ether (1/1, 8 mL) was introduced, the mixture was filtered on Celite, the solvent removed by evaporation and the crude purified by silica gel chromatography (elution with cyclohexane/ethyl acetate, 6/4) to afford 60 mg of the desired lactone (61%, yield).

MS (APCI+) m/z 829 (M+H⁺): 100%.

### Step 2: 1-C-Methylene 2,3,4,6-tetra-O-benzyl-7-(dibenzyloxyphosphoryl)-D-glycero-D-manno-heptopyranose

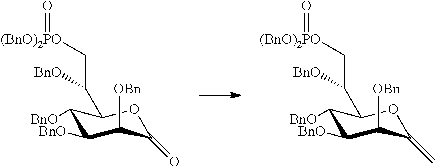

To a solution of the previous lactone (800 mg, 970 μmol) in dry toluene, Petasis reagent (26.77 mL, 5.79 mmol) was slowly added at room temperature and the mixture was stirred two hours at 70° C. Then, the reaction was cooled down to room temperature, the solvent was removed by evaporation and the crude reaction mixture was purified by silica gel chromatography (ethyl acetate/cyclohexane:3/7) to give the desired product (606 mg, 76% yield).

MS (ESI+) m/z 849 [M+Na]⁺: 100%.

### Step 3: 1-C-Hydroxymethylene 2,3,4,6-tetra-O-benzyl-7-(dibenzyloxyphosphoryl)-D-glycero-D-manno-heptopyranose

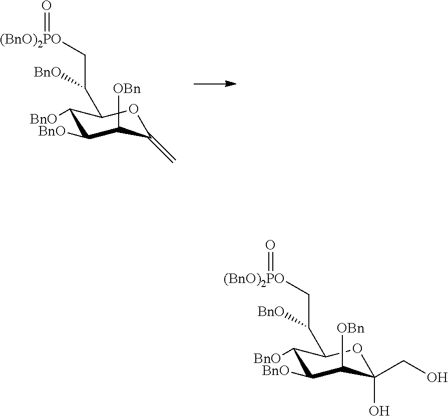

A mixture of the previous intermediate (100 mg, 240 μmol), K₂OsO₄ (26 mg, 72 μmol), K₃Fe(CN)₆ (221 mg, 672 μmL), t-BuOH (1.2 mL), H₂O (1.2 mL), toluene (0.5 mL) and K₂CO₃ (100 mg) was stirred at room temperature overnight. Then, Na₂SO₃ (420 mg) was added and the reaction mixture was stirred for 1 hour. Then, 20 mL of water were added, and the mixture was extracted with EtOAc (3×50 mL). The organic layer was dried over MgSO₄, filtered and the solvent were removed under vacuum. The crude was purified by silica gel chromatography (elution with ethyl acetate/cyclohexane 4/6) to give the desired product (100 mg, 42% yield).

MS (ESL) m/z 883 [M+Na]⁺: 100%.

### Step 4: 1-deoxy-1-β-C-Acetoxymethylene 2,3,4,6-tetra-O-benzyl-7-(dibenzyloxyphosphoryl)-D-glycero-D-manno-heptopyranose

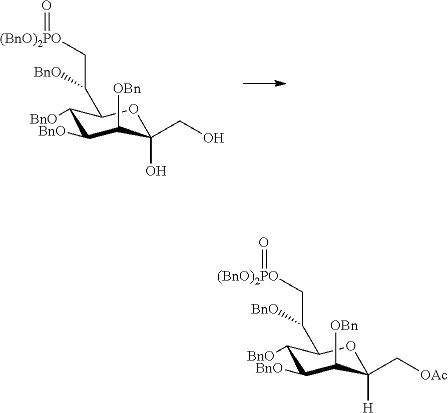

A mixture of the previous intermediate (100 mg, 115 μmol), Ac₂O (70 μL, 690 μmol), 4-DMAP (1 mg, 11.5 μmol) and dry pyridine (1 mL) was stirred at room temperature overnight. Then the reaction quenched by the addition of brine (10 mL) and extracted with DCM (30 mL×3). The organic layer dried, filtered and the solvent were removed by evaporation. The resulting crude was directly dissolved in dry DCM (2 mL). Then, BF₃.THF (44 μL, 287 μmol) and Et₃SiH (54 μL, 287 μmol) were added at T=0° C. and the reaction stirred at room temperature for 3 hours. Then, a saturated solution of NaHCO₃ (10 mL) was added and the mixture extracted with DCM (30 mL×3). The organic layers were dried, filtered and the solvent were removed by evaporation. The crude was purified by silica gel chromatography, eluted with ethyl acetate/cyclohexane (4/6) to give the desired compound (83 mg, 82% yield).

MS (ESI+) m/z 909 [M+Na]⁺: 100%.

NOE measurements display a correlation between H-1/3 as well as another correlation between H-1/5 therefore confirming the beta anomeric assignment.

### Step 5: 1-β-C-Hydroxymethylene 1-Deoxy-7-O-phosphoryl-D-glycero-D-manno-heptopyranose

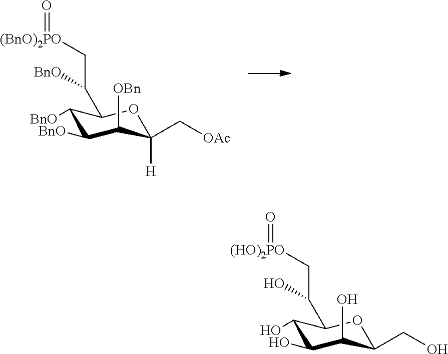

A solution of the previous intermediate (20 mg, 23 μmol) and methylamine (1.2 mL, 33% in ethanol) was stirred at room temperature overnight. Then, 10 mL of ethyl acetate and 10 mL of NH₄Cl (1 M) were added and the mixture was extracted with ethyl acetate (10 mL×3). The organic layers were dried, filtered and the solvent was removed by evaporation. The crude was purified by silica gel chromatography, eluted with a mixture ethyl acetate/cyclohexane (4/6) to give the corresponding alcohol. The latter was dissolved in EtOAc/EtOH/H₂O (1.5/0.9/0.6 mL) and Pd/C (10°/0, 130 mg) was added. This suspension was stirred at room temperature under hydrogen atmosphere for 48 hours. The crude was purified by HPLC Zorbax SB-C18, semi-preparative column, H₂O as isocratic elution) yielding 3 mg of the desired product (43% yield).

¹H NMR (100 Hz, D₂O, 25° C.) δ 4.03 (m, 1H, H-6), 3.90 (m, 1H, H-7a), 3.78-3.69 (m, 2H, H-2 and H-7b), 3.62-3.48 (m, 5H, H-1, H-3, H-4, 2×H^{CH2}), 3.52 (d, 1H, H-5 J=7.8 Hz).

¹³C NMR (100 Hz, D₂O, 25° C.) δ (ppm) 80.5 (C-5), 78.9 (C-1), 74.3 (C-3), 70.9 (d, J=6.7 Hz, C-6), 69.2 (C-2), 67.5 (C-4), 64.5 (d, J=3.8 Hz, C-7), 61.6 (CH₂).

³¹P NMR (D₂O, 101 MHz) δ 3.66.

HRMS (ESI−) for C₈H₁₆O₁₀P (M−H⁺) meas. 303.0472 calc. 304.0481.

### Example 12

### 1-C-Methyl 7-O-phosphoryl-D-glycero-α-D-manno-heptopyranose

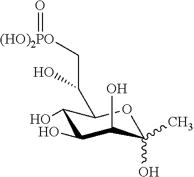

A solution of 1-C-Methylene 2,3,4,6-tetra-O-benzyl-7-(dibenzyloxyphosphoryl)-D-glycero-D-manno-heptopyranose (Example 11, step 2, 30 mg, 36 μmol), Pd/C (10%, 100 mg) in EtOAc/EtOH/H₂O (3/1.8/1.2 mL) was stirred at room temperature under hydrogen atmosphere. After 48 hours, the reaction mixture was filtered over celite, washed with water and lyophilized. The crude mixture was purified by HPLC (Zorbax SB-C18, isocratic elution with water) yielding 4.0 mg of the desired product (36% yield).

¹H NMR (100 Hz, D₂O, 25° C.) δ 3.95 (m, 1H, H-6), 3.84-3.82 (m, 1H, H-7a), 3.76-3.68 (m, 3H, H-3, H-5, H-7b), 3.62 (d, 1H, H-4, J=9.6 Hz), 3.55 (d, 1H, J=2.7 Hz, H-2), 1.30 (s, 3H, CH₃).

¹³C NMR (100 Hz, D₂O, 25° C.) S (ppm) 97.8 (C-1), 72.7 (C-2), 72.6 (C-5), 71.3 (d, J=6.7 Hz, C-6), 71.0 (C-3), 67.1 (C-4), 65.2 (d, J=4.8 Hz, C-7), 24.4 (CH₃).

³¹P NMR (D₂O, 101 MHz) δ4.84.

HRMS (ESI−) for C₈H₁₆O₁₀P (M−H⁺) meas. 303.0484 calc. 304.0481.

### Example 13

### 1-C-Hydroxymethylene 7-O-phosphoryl-D-glycero-D-manno-heptopyranose

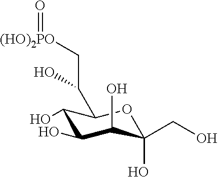

A solution of 1-C-Hydroxymethylene 2,3,4,6-tetra-O-benzyl-7-(dibenzyloxyphosphoryl)-D-glycero-D-manno-heptopyranose (Example 11, step 3, 20 mg, 23 μmol), Pd/C (10%, 84 mg) in EtOAc/EtOH/H₂O: 1.5/0.9/0.6 mL was stirred at room temperature under an hydrogen atmosphere. After 48 hours, the reaction mixture was filtered over Celite, washed with water and lyophilized. The crude was purified by HPLC (Zorbax SB-C18, semi-preparative column, H₂O as isocratic elution) yielding 4.0 mg of the desired product (57%).

¹H NMR (100 Hz, D₂O, 25° C.) δ 3.96 (m, 1H, H-6), 3.88 (m, 1H, H-7a), 3.77-3.69 (m, 4H, H-2, H-3, H-5, H-7b), 3.63 (d, 1H, J=9.6 Hz, H-4), 3.52 (d, J=11.9 Hz, 1H, CH₂), 3.40 (d, J=11.9 Hz, 1H, CH₂).

¹³C NMR (100 Hz, D₂O, 25° C.) δ (ppm) 97.8 (C-1), 72.6 (C-5), 71.2 (C-3 and C-6), 69.9 (C-2), 67.6 (C-4), 65.1 (C-7), 64.1 (CH₂).

³¹P NMR (D₂O, 101 MHz) δ 2.96

HRMS (ESI—) for C₈H₁₆O₁₁P Meas. 319.0419 calc. 319.0430.

### Example 14

### D-Glycero-α-D-manno-oct-2-ulopyranose

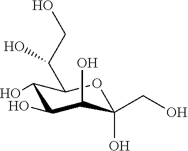

### Step 1: Phenyl 2,3,4,6,7-penta-O-benzyl-1-thio-D-glycero-α-D-manno-heptopyranoside

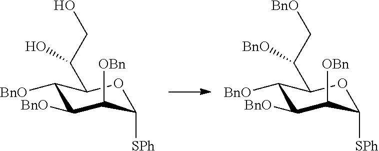

A mixture of phenyl 2,3,4-tri-O-benzyl-D/L-glycero-α-D-manno-heptopyranoside (see example 2, step 4, 385 mg, 0.67 mmol) was dissolved in DMF (9 mL) under argon and cooled in an ice-water bath. Then, NaH 60% (4 equiv., 2.7 mmol, 0.107 g) was added. After a few minutes, benzyl bromide (4 equiv., 2.7 mmol, 0.32 mL) was added dropwise over a 10 min period. The mixture was stirred at room temperature until complete conversion (1.5 h), as shown by TLC (4/1 hexane/EtOAc). Then cold water (15 mL) was added and the mixture was extracted with diethyl ether (3×4 mL). The combined organic phases were washed with brine and water and then dried with MgSO₄. After filtration and evaporation of the solvent under vacuum, the residue was purified by column chromatography on silica-gel (from EtOAc/Hexane, 1:11 to EtOAc/Hexane, 1:10) to afford the D,D-fully benzylated compound as colourless oil (91 mg, 18%).

R_{f}=0.30 (EtOAc/Hexane, 1:10);

¹H NMR (CDCl₃, 400 MHz) δ: 7.53-7.13 (m, 30H, Ph), 5.54 (br. d, 1H, H-1, J=1.9 Hz), 4.87 (d, 1H, part A of AB system, H-a, CH₂Ph, J=10.5 Hz), 4.77 (d, 1H, part A of AB system, H-a, CH₂Ph, J=10.5 Hz), 4.70-4.58 (m, 6H, CH₂Ph), 4.49 (d, 1H, part A of AB system, H-a, CH₂Ph, J=11.9 Hz), 4.44 (d, 1H, part B of AB system, H-b, CH₂Ph), 4.41 (dd, 1H, H-5, J_{4,5}=9.6, J_{5,6}=1.1 Hz), 4.14 (t, 1H, H-4), 4.03 (ddd, 1H, H-6), 3.97 (dd, 1H, H-2), 3.87 (dd, 1H, H-3, J_{2,3}=3.0 Hz), 3.79 (dd, part A of ABX system, 1H, H-7a, J_{6,7a}=4.4, J_{7a,7b}=10.4 Hz), 3.72 (dd, part B of ABX system, 1H, H-7b, J_{6,7b}=6.5 Hz).

¹³C NMR (CDCl₃, 100 MHz) δ: 138.9, 138.6, 138.4, 138.3, 138.1, 134.5 (Cq, Ph), 132.1, 129.1, 128.6, 128.5, 128.4, 128.4, 128.3, 128.1, 128.1, 127.8, 127.8, 127.7, 127.6, 127.6, 127.6, 127.4 (CH, Ph), 85.9 (C-1), 80.6 (C-3), 78.8 (C-6), 76.6 (C-2), 75.0 (CH₂Ph), 74.9 (C-4), 73.4 (CH₂Ph), 73.2 (C-5), 72.5, 72.3, 72.1 (3×CH₂Ph), 71.2 (C-7).

HRMS (ESI⁺): calcd for C₄₈H₄₈O₆S [M+Na]⁺775.3064. found 775.3064.

### Step 2: 2,3,4,6,7-Penta-O-benzyl-D-glycero-D-manno heptopyranose

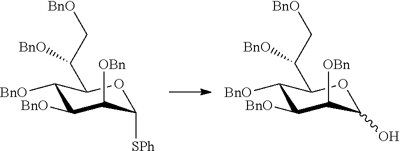

To a solution of the previous phenyl thioglycoside (145 mg, 0.19 mmol) in acetone (3.4 mL) and water (0.7 mL) at −5° C., N-bromosuccinimide (NBS, 0.1 g, 3 equiv, 0.6 mmol) was added in absence of light. After 20 min at −5-0° C., TLC (hexane/EtOAc, 4:1) showed complete conversion and the mixture was quenched with a saturated NaHCO₃ soln, diluted with EtOAc, washed with saturated Na₂S₂O₃ and water. The combined aqueous phases were extracted with EtOAc. The organic layer was dried with MgSO₄ and filtered. After concentration under vacuum, the residue was purified by column chromatography on silica gel (hexane/EtOAc, 4:1) to yield the title compound as colourless oil (115 mg, 91%) in an α/β ratio of 1:0.3.

¹H NMR (CDCl₃, 400 MHz) δ: 7.39-7.12 (m, Ph), 5.20 (brd, H-1α, J=1.9 Hz), 5.03 (d, part A of AB system, H-a, CH₂Ph, β-anomer), 4.88-4.44 (m, CH₂Ph, H-1β), 4.13-3.92 (m, H-3α, H-4α, H-5α, H-6α), 3.85-3.67 (m, H-2α, H-2β, CH₂-7α, CH₂-7β), 3.66-3.57 (m, H-3β).

¹³C NMR (CDCl₃, 100 MHz) δ: 138.9, 138.7, 138.6, 138.6, 138.6, 138.4, 138.0 (Cq, Ph), 128.6, 128.6, 128.5, 128.4, 128.4, 128.4, 128.3, 128.1, 128.0, 127.9, 127.7, 127.7, 127.7, 127.6, 127.6, 127.5 (CH, Ph), 93.8 (C-1β), 92.6 (C-1α), 82.9 (C-3β), 80, 2 (C-3α), 77.9 (C-4α), 75.4 (C-2α), 75.0, 74.8, 73.4, 72.7, 72.6, 72.4, 72.3 (5×CH₂Ph, C-5, C-6), 70.7 (C-7β),70.7 (C-7α).

HRMS (ESI+): calcd for C₄₂H₄₄O₇ [M+Na]⁺683.2979. found 683.2879.

### Step 3: 2,3,4,6,7-Penta-O-benzyl-D-glycero-D-manno-heptono-δ-lactone

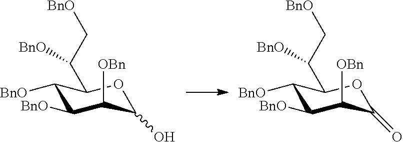

To a solution of the previous lactol (26 mg, 0.04 mmol) in anhydrous dichloromethane (1.5 mL) under argon, previously activated 4 Å molecular sieves (45 mg) were added and the mixture was stirred at room temp. for ca. 20 min. Then PCC (76 mg, 9 equiv., 0.35 mmol) was added and the whole mixture was stirred at room temp. until complete conversion, as indicated by TLC (ca. 5 h, 1:4, EtOAc/Hexane). The mixture was triturated with Et₂O/EtOAc (3×10 mL, 1:1), and was filtered through Celite. The eluate was concentrated and the residue was purified by column chromatography on silica gel (hexane/EtOAc, 4:1) to afford the desired aldonolactone as colourless oil (24 mg, 93%).

¹H NMR (CDCl₃, 400 MHz) δ: 7.41-7.20 (m, 25H, Ph), 5.05 (d, part A of AB system, 1H, H-a, CH₂Ph, J=12.3 Hz), 4.78 (d, part A of AB system, 1H, H-a, CH₂Ph, J=12.3 Hz), 4.66 (br. s, 2H, CH₂Ph), 4.62-3.55 (m, 2H, CH₂Ph), 4.46 (d, part A of AB system, 1H, H-a, CH₂Ph, J=11.9 Hz), 4.42 (d, part B of AB system, 1H, H-b, CH₂Ph), 4.39-4.34 (m, 2H, H-2, H-5), 4.31 (d, part A of AB system, 1H, H-a, CH₂Ph, J=11.5 Hz), 4.26 (d, part B of AB system, 1H, H-b, CH₂Ph), 4.08 (dd, 1H, H-4, J_{3,4}=1.5, J_{4,5}=6.2 Hz), 4.02 (br. t, 1H, H-3), 3.87-3.89 (m, 1H, H-6), 6.63-6.59 (m, 2H, CH₂-7).

¹³C NMR (CDCl₃, 100 MHz) δ: 169.7 (C-1), 138.3, 138.2, 137.9, 137.5, 137.2 (Cq, Ph), 128.6, 128.6, 128.5, 128.5, 128.4, 128.2, 128.1, 128.0, 127.9, 127.8 (CH, Ph), 79.9, 77.9, 76.7 75.7, 75.3 (C-2, C-3, C-4. C-5, C-6), 73.5, 73.1, 73.1, 71.7 (CH₂Ph), 69.2 (C-7).

HRMS (ESI⁺): calcd for C₄₂H₄₂O₇ [M+H]⁺659.3003. found 659.3000.

### Step 4: 2,6-Anhydro-3,4,5,7,8-penta-O-benzyl-1-deoxy-D-glycero-D-manno-oct-1-enitol

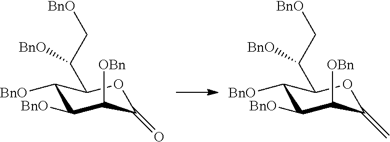

To solution of the previous aldonolactone (60 mg, 0.09 mmol) in anhydrous toluene (2 mL) and was added dimethyl titanocene (soln. 5% in toluene, 1.1 mmol, 5 mL) in the absence of light under argon. The solution was stirred overnight at 65° C. in the dark. The brownish solution was concentrated under vacuum. The residue was dissolved in a minimum of toluene and subjected to column chromatography on silica gel (hexane/EtOAc, 9:1) to afford the desired exoglycal as a colourless oil (46 mg, 77%).

¹H NMR (MeOD, 600 MHz) δ: 7.42-7.10 (m, 25H, Ph), 4.78 (d, part A of AB system, 1H, H-a, CH₂Ph, J=11.4 Hz), 4.75 (s, 1H, H-1a), 4.72-4.58 (m, 4H, CH₂Ph), 4.57-4.48 (m, 3H, H-1b, CH₂Ph), 4.44-4.37 (m, 3H, CH₂Ph, H-b, Bn), 4.24-4.15 (m, 2H, H-3, H-5, J_{3,4}=3.0, (J_{4,5}=J_{5,6}=8.5 Hz), 4.00 (ddd, 1H, H-7), 3.76-3.59 (m, 4H, H-4, H-6, CH₂-8).

¹³C NMR (MeOD, 150 MHz) δ: 156.6 (C-2), 139.8, 139.7, 139.6, 139.6, 139.5 (Cq, Ph), 129.4, 129.4, 129.3, 129.3, 129.3, 129.1, 129.1, 129.1, 128.9, 128.8, 128.6, 128.6 (CH, Ph), 99.0 (C-1), 82.8, 81.7 (C-4, C-6), 79.6 (C-7), 75.5 (C-3), 75.4 (CH₂Ph), 75.0 (C-5), 74.2, 73.5, 72.4, 71.3 (4×CH₂Ph), 71.0 (C-8).

### Step 5: 3,4,5,7,8-Penta-O-benzyl-D-glycero-α-D-manno-oct-2-ulopyranose

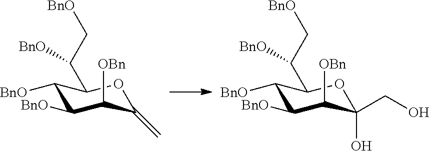

To a solution of the previous exoglycal (36 mg, 0.06 mmol) in THF/H₂O (1.5 mL, 2:1) was added N-methylmorpholine-N-oxide (2 equiv., 0.11 mmol, 13 mg). After stirring at room temp. for 10 min, osmium tetraoxide (cat. amount) was added and the mixture was stirred at room temp. until complete conversion as observed by TLC (1 h, EtOAc/Hexane, 1:1). Then a saturated Na₂S₂O₅ solution (2 mL) was added. The mixture was extracted with EtOAc (3×3 mL). The organic phase was washed with aq. HCl 1N soln. (3 mL), then saturated aqueous NaHCO₃ solution (3 mL) and brine (5 mL), and then it was dried with MgSO₄. After filtration and evaporation, the crude was purified by column chromatography on silica-gel (hexane/EtOAc, 3:2) to afford the diol 8 as a colourless oil (30 mg, 80%);

[α]_{D}²²=+27.9 (c=0.5,in MeOH).

¹H NMR (MeOD, 600 MHz) δ: 7.49-7.34 (m, 4H, Ph), 7.32-7.19 (m, 19H, Ph), 7.15-7.11 (m, 2H, Ph), 4.92 (d, part A of AB system, 1H, H-a, Bn, J=11.2 Hz), 4.82 (d, part A of AB system, 1H, H-a, Bn, J=10.8 Hz), 4.78-4.64 (m, 4H, CH₂Ph), 4.69 (d, part B of AB system, 1H, H-b, Bn, J=11.2 Hz), 4.52 (d, part B of AB system, 1H, H-b), 4.38 (d, part A of AB system, 1H, H-b, Bn, J=11.9 Hz), 4.35 (d, part B of AB system, 1H, H-b), 4.12 (dd, 1H, H-4, J_{3,4}=2.6, J_{4,5}=8.6 Hz), 4.08-4.01 (m, 3H, H-3, H-5, H-6), 3.94 (dd, 1H, H-7), 3.69 (d, 1H, part A of AB system, H-1a, J_{a,b}=11.2 Hz), 3.65 (part A of ABX system, 1H, H-8a, J_{7,8a}=4.4, J_{8a,8b}=110.8 Hz), 3.61 (part B of ABX system, 1H, H-8b, J_{7,8b}=7.0 Hz), 3.52 (d, 1H, part B of AB system, H-1b).

¹³C NMR (MeOD, 75 MHz) δ: 140.5, 140.0, 139.9, 139.8, 139.7 (Cq, Ph), 129.4, 129.3, 129.2, 129.1, 128.9, 128.8, 128.6, 128.5, 128.5, 128.5, 128.4 (CH, Ph), 99.3 (C-2), 83.1 (C-4), 80.0 (C-7), 76.8 (C-3), 76, 2 (C-5), 75.7 (CH₂Ph), 75.6 (CH₂Ph), 75.0 (C-5), 74.1 (C-6), 74.1, 73.6, 73.0 (3×CH₂Ph), 71.9 (C-8), 66.1 (C-1).

### Step 6: D-Glycero-α-D-manno-oct-2-ulopyranose

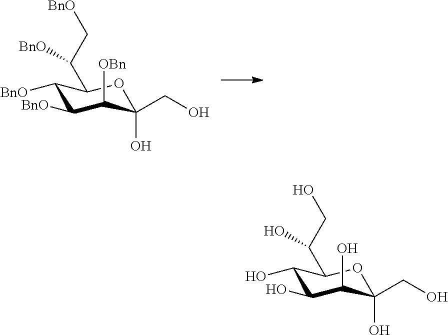

The previous benzylated octulose (26 mg, 0.038 mmol) was dissolved in anhydrous methanol (2 mL). Then a catalytic amount (one spatula tip) of 10% Pd/C was added and the reaction mixture was stirred under a hydrogen atmosphere for 48 h at rt. The mixture was then filtered, washed with MeOH (3×) and concentrated in vacuum. The crude was dissolved in water (HPLC grade) and subjected to gel filtration using a PD-10 Sephadex G 25 column (water as eluent). The eluate was lyophilized yielding the title compound as a solid (8.3 mg, 92%);

[α]_{D}²⁰=+5.2 (c=0.2, in MeOH).

¹H NMR (MeOD, 600 MHz) δ: 3.90 (dt, 1H, H-7, J_{6,7}=J_{7,8a}=3.8, J_{7,8b}=6.3 Hz), 3.85 (dd, 1H, H-4, J_{3,4}=3.3, J_{4,5}=8.7 Hz), 3.78 (dd, 1H, H-3, J_{3,4}=3.3 Hz), 3.77-3.74 (m, 3H, H-5, H-6, H-8a), 3.68 (part B of ABX system, 1H, H-8b, J_{8a,8b}=11.7 Hz), 3.60 (d, 1H, part B of AB system, H-1a, J_{a,b}=11.4 Hz), 3.56 (d, 1H, part B of AB system, H-1b).

¹³C NMR (MeOD, 600 MHz) δ: 98.75 (C-2), 74.82 (C-7), 73.95 (C-6), 73.08 (C-4), 72.36 (C-3), 70.19 (C-5), 66.39 (C-1), 63.98 (C-8).

NOESY spectrum does not display any NOE correlation signal between H-3 or H-5 and the CH₂ from the C-glycoside therefore confirming the beta CH₂OH anomeric assignment.

### Example 15

### 1,5-Anhydro-D-glycero-D-gluco-heptitol

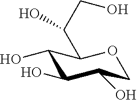

### Step 1: 1,2,4,6,7-Penta-O-acetyl-3-O-benzyl D-glycero-D-gluco-heptopyranose

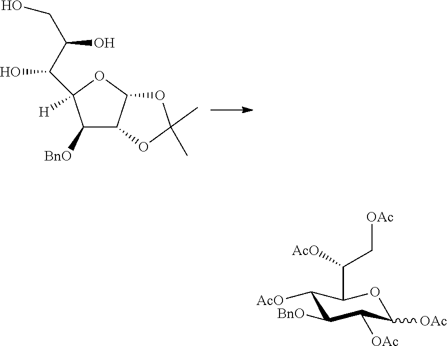

A solution of (1R,2R)-1-[(3aR,5R,6S,6aR)-6-(benzyloxy)-2,2-dimethyltetrahydrofuro[2,3-d][1,3]dioxol-5-yl]propane-1,2,3-triol (583 mg, 1.71 mmol, prepared according to J. S. Brimacombe, A. K. M. S. Kabir, Carbohydr. Res. 1986 (150) 35-51.) in 50% aqueous TFA (10 mL) was stirred at room temperature for 16 h. The reaction mixture was concentrated and coevaporated with toluene (3×10 mL). The remaining slightly red oil was taken up in pyridine (4 mL), cooled to 0° C. and Ac₂O (4 mL) was added dropwise at 0° C. The reaction mixture was stirred at room temperature for 12 h, then cooled to 0° C. and MeOH (5 mL) was added dropwise. The reaction mixture was diluted with CHCl₃ (15 mL), saturated aqueous NaHCO₃ (10 mL) was added and the reaction mixture was stirred for 15 min. The layers were separated, the aqueous layer was re-extracted with CHCl₃ (10 mL) and the combined organic layers were dried (MgSO₄) and concentrated to dryness. The residue was purified by column chromatography (eluent: hexane/EtOAc, 3:1→EtOAc) to afford the title compound (645 mg, 73%) as a white solid.

¹H NMR for α anomer (600 MHz, CDCl₃): δ 7.36-7.21 (m, 5H, Ph), 6.29 (d, 1H, J_{1,2}=3.5 Hz, H-1), 5.13 (t, 1H, J_{4,3}=9.8 Hz, H-4), 5.11 (bs, 1H, H-6), 5.00 (dd, 1H, J_{2,3}=9.9 Hz, H-2), 4.68 (d, 1H, CH₂Ph), 4.60 (d, 1H, CH₂Ph), 4.32 (dd, 1H, J_{7a,7b}=12.0 Hz, J_{7a,6}=3.9 Hz, H-7a), 4.20 (dd, 1H, J_{7b,6}=7.5 Hz, H-7b), 4.04 (dd, 1H, J_{5,6}=10.5 Hz, H-5), 3.92 (t, H-3), 2.14, 2.06, 2.04, 2.01, 1.98 (5×s, 3H, OAc).

¹³C (150 MHz, CDCl₃): δ 128.46, 127.88, 127.56 (CH₂Ph), 89.23 (C-1), 77.2 (C-3, covered by CHCl₃ signal), 74.84 (CH₂Ph), 71.71 (C-5), 71.32, 69.91, 69.83 (C-2,4,6), 61.27 (C-7), 20.90-20.67 (5×OAc). ¹H NMR for β anomer (600 MHz, CDCl₃): δ 7.36-7.21 (m, 5H, Ph), 5.61 (d, 1H, J_{1,2}=7.9 Hz, H-1), 5.16-5.08 (m, 3H, H-2,4,6), 4.60 (s, 2H, CH₂Ph), 4.29 (dd, 1H, J_{7a,6}=4.0 Hz, J_{7a,7b}=12.0 Hz, H-7a), 4.25 (dd, 1H, J_{7b,6}=7.1 Hz, H-7b), 3.77 (dd, 1H, J_{5,6}=9.9 Hz, H-5), 3.71 (t, 1H, J_{3,4}=8.9 Hz, H-3), 2.10, 2.07, 2.05, 2.02, 1.97 (5×s, 3H, OAc).¹³C (150 MHz, CDCl₃): δ 128.51, 127.98, 127.84 (CH₂Ph), 92.00 (C-1), 79.88 (C-3), 74.39 (C-5), 74.21 (CH₂Ph), 71.36 (C-2), 69.85, 69.83 (C-4, C-6), 61.25 (C-7), 20.90, 20.84, 20.78, 2 0.72, 20.67 (5×OAc).

HR MS: C₂₅H₃₁O₁₄ [M+COOH]— calc: 555.1719. found 555.1714.

### Step 2: 1,2,3,4,6,7-Hexa-O-acetyl-D-glycero-D-gluco-heptopyranose

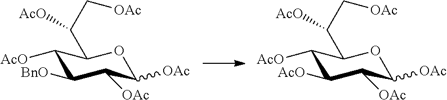

The previous compound (633 mg, 1.24 mmol) was dissolved in MeOH (24 mL) and hydrogenated in an H-Cube for 12 h (H-Cube SS; cartridge: Pd/C 33 mm; solvent: MeOH; flow rate: 0.2 mL; H₂-mode: full; temperature: 50° C.). The reaction mixture was concentrated (540 mg) and dissolved in pyridine (2 mL). Ac₂O (500 μA and a catalytic amount of DMAP were added and the reaction was stirred at room temperature for 12 h. The reaction mixture was cooled to 0° C., MeOH (1 mL) was added and the reaction mixture was stirred for 10 min and then diluted with DCM (5 mL). The organic phase was washed with saturated aqueous NaHCO₃ (2×5 mL), dried (MgSO₄) and evaporated to dryness. The residue was purified by column chromatography (silica gel 60, toluene/EtOAc4:1→toluene/EtOAc 1:1) to give the title compound (557 mg, 1.20 mmol, 97%) as white solid.

¹H NMR (600 MHz, CDCl₃) for α anomer: δ 6.31 (d, 1H, J_{1,2}=3.7 Hz, H-1), 5.43 (t, 1H, J=9.6 Hz, H-3), 5.18-5.15 (m, 2H, H-4, H-6), 5.04 (dd, 1H, H-2), 4.32 (dd, 1H, J_{7a,6}=4.26 Hz, J_{7a,7b}=12 Hz, H-7a), 4.16 (dd, 1H, J_{5,6}=10.5 Hz, J_{5,4}=2.8 Hz, H-5), 4.14 (dd, 1H, J_{7b,6}=7.1 Hz, H-7b), 2.17, 2.16, 2.08, 2.08, 2.05, 2.02 (6×s, each 3H, 6 OAc). ¹³C NMR (150 MHz, CDCl₃): δ. 88.78 (C-1), 70.96 (C-5), 69.92 (C-3), 69.75 (C-6), 69.01 (C-2), 68.81 (C-4), 61.38 (C-7), 20.85-20.51 (6×OAc).

¹H NMR (600 MHz, CDCl₃) for β anomer: δ 5.68 (d, 1H, J_{1,2}=8.1 Hz, H-1), 5.21 (t, 1H, J_{3,2}=J_{3,4}=9.2 Hz, H-3), 5.18-5.15 (m, 1H, H-6), 5.13 (t, 1H, H-4), 5.08 (dd, 1H, H-2), 4.29 (dd, 1H, J_{7a,6}=4.26 Hz, J_{7a,7b}=11.9 Hz, H-7a), 4.19 (dd, 1H, J_{7b,6}=7.0 Hz, H-7b), 3.88 (dd, 1H, J_{5,6}=9.9 Hz, J_{5,4}=3.1 Hz, H-5), 2.11, 2.08, 2.07, 2.05, 2.03, 2.01 (6×s, each 3H, 60Ac).

¹³C NMR (150 MHz, CDCl₃): 91.69 (C-1), 73.87 (C-5), 72.79 (C3), 70.05 (C-2), 69.65 (C-6), 68.78 (C4), 61.28 (C-7), 20.85-20.51 (6×OAc). HR MS: C₁₉H₂₆O₁₃S [M+Na]⁺ calc: 485.1266. found 485.1268.

### Step 3: Phenyl 2,3,4,6,7-penta-O-acetyl-1-thio-D-glycero-α,β-D-gluco-heptopyranoside

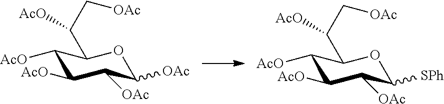

A solution of previous intermediate (557 mg, 1.20 mmol) in anhydrous DCM (5 mL) was stirred under argon at 0° C. Thiophenol (143 μL, 1.20 mmol) was added followed by dropwise addition of a 1M solution of SnCl₄ in DCM (663 μL) and the solution was stirred at room temperature for 12 h. The reaction mixture was diluted with DCM (5 mL), washed with saturated aqueous NaHCO₃ (5 mL) and the aqueous phase was re-extracted with DCM (5 mL). The combined organic phases were dried (MgSO₄), evaporated to dryness and the residue was directly purified by column chromatography (silica gel, toluene/EtOAc 7/1) to afford an anomeric mixture (α:β=1:2) of the title compound (266 mg, 0.51 mmol, 43%) as colorless oil.

¹H NMR (600 MHz, CDCl₃) for α anomer: δ 7.51-7.15 (m, 5H, SPh), 6.27 (d, 1H, J_{1,2}=4.1 Hz, H-1), 5.51 (t, 1H, J_{3,2}=_{3,4}=9.6 Hz, H-3), 5.22 (m, 1H, H-6), 5.19-5.17 (m, 1H, H-4), 4.96 (dd, 1H, H-2), 4.35 (dd, 1H, J_{5,4}=10.4 Hz, J_{5,6}=2.3 Hz, H-5), 4.29 (dd, 1H, J_{7a,6}=4.6 Hz, J_{7a,7b}=12 Hz, H-7a), 4.17 (dd, 1H, J_{7b,6}=7.2 Hz, H-7b), 2.09 (s, 9H), 2.06 (s, 3H) and 2.03 (s, 3H, 5 OAc).

¹H NMR (600 MHz, CDCl₃) for β anomer: δ 7.51-7.15 (m, 5H, SPh), 5.20-5.13 (m, 2H, H-3,H-6), 5.04 (t, 1H, J_{4,3}˜_{4,5}˜10 Hz, H-4), 4.92 (t, 1H, J_{2,1}=10.0 Hz, _{2,3}=9.6 Hz, H-2), 4.64 (d, 1H, J_{1,2}=10.0 Hz, H-1), 4.32 (dd, 1H, J_{7a,6}=4.2 Hz, J_{7a,7b}=11.8 Hz, H-7a), 4.21 (dd, 1H, J_{7b,6}=7.2 Hz, H-7b), 3.74 (dd, 1H, J_{5,6}=10.2, J_{5,4}=2.7 Hz, H-5), 2.09, 2.07, 2.05, 2.03, 1.98 (5×s, 3H, 50Ac). HR MS: C₂₃H₂₈O₁₁S [M+Na]⁺ calc: 535.1245. found 535.1247.

### Step 4: 2,3,4,6,7-Penta-O-acetyl-1,5-anhydro-D-glycero-D-gluco-heptitol

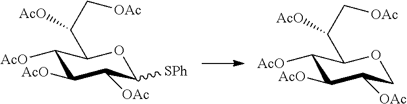

The previous intermediate (53 mg, 103 μmol) was dissolved in EtOH (50 mL) and dethionated in an H-Cube for 32 h. (H-Cube SS; cartridge:
Raney-N±33 mm; solvent: EtOH; flow rate: 0.2 mL; H₂-mode: full; temperature: 40° C.). The reaction mixture was evaporated to dryness and directly purified by column chromatography (silica gel, toluene/acetone 14:1) to give the title compound (23 mg, 57 μmol, 55%) as colorless oil;

[α]_{D}²⁰=+37.3° (c 1.2, CHCl₃).

¹H NMR (600 MHz, CDCl₃): δ 5.18-5.13 (m, 2H, H-3,H-6), 5.04 (t, 1H, J_{2,3}=J_{3,4}=9.9 Hz, H-3), 4.95 (dt, 1H, J_{2,1a}=9.9 Hz, J_{2,1b}=5.5 Hz, H-2), 4.31 (dd, 1H, J_{7a,6}=4.1 Hz, J_{7a,7b}=11.9 Hz, H-7a), 4.17 (dd, J_{7b,6}=7.5 Hz, H-7b), 4.14 (dd, 1H, J_{1a,1b}=11.2 Hz, H-1a), 3.62 (dd, 1H, J_{5,4}=10.1 Hz, H-5), 3.26 (t, 1H, H-1b), 2.09, 2.07, 2.04, 2.02, 2.02 (5×s, each 3H, 5 OAc).

¹³C NMR (150 MHz, CDCl₃): δ 170.55, 170.24, 169.92, 169.75, 169.57 (5×CO), 77.96 (C-5), 73.68 (C-3), 69.95 (C-6), 69.03 (C-4), 68.74 (C-2), 66.77 (C-1), 61.41 (C-7), 20.90-20.65 (5×OAc).

HR MS: C₁₇H₂₄O₁₁ [M+Na]⁺ calc: 427.1211. found 427.1214.

### Step 5: 1,5-Anhydro-D-glycero-D-gluco-heptitol

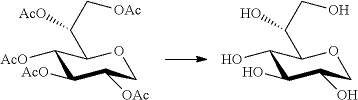

A solution of NaOMe in MeOH (100 μL, 0.1 M) was added to a solution of previous intermediate (23.6 mg, 58 μmol) in MeOH (2 mL) at rt and the reaction was stirred for 4 h. The reaction mixture was then neutralized (Dowex 50H⁺ form), filtered and the filtrate was concentrated. The residue was taken up in HPLC grade H₂O and purified over a short PD10 column (Sephadex G-25, 1.45×5.0, 8.3 mL column volume, eluent: water). Product containing fractions were pooled and lyophilized to give the target compound (10.5 mg, 92%) as an amorphous solid;

[α]_{D}²⁰=+28.2° (c 0.5, H₂O).

¹H NMR (600 MHz, D₂O): δ 3.96 (dt, 1H, J_{6,7a}=3.4 Hz, J_{6,7b}=7.5 Hz, H-6), 3.92 (dd, 1H, J_{1a,1b}=11 Hz, J_{1a,2}=5.4 Hz, H-1a), 3.72 (dd, 1H, J_{7a,7b}=12.0 Hz, H-7a), 3.63 (dd, 1H, H-7b), 3.53 (ddd, 1H, J_{2,3}˜10.5 Hz, H-2), 3.43 (t, 1H, J_{4,3=} J_{4,5}=9.3 Hz, H-4), 3.39-3.35 (m, 2H, H-3, H-5), 3.19 (d, 1H, J_{1b,2}=10.9 Hz, H-1b).

¹³C NMR (150 MHz D₂O): δ 80.62 (C-5), 77.56 (C-3), 71.59 (C-6), 70.13 (C-4), 69.05 (C-2), 68.91 (C-1), 61.43 (C-7).

HR MS: C₇H₁₄O₆ [M+Na+] calc: 217.0683. found 217.0682.

Pharmacological Study of the Compounds of the Invention

Inhibition of the Enzymatic Activity of GmhA (Luminescent Assay):

The assay buffer “AB” contained 50 mM Hepes pH7.5, 1 mM MnCl₂, 25 mM KCl, 0.012% Triton-X100 and 1 mM dithiothreitol (DTT) and 0.1 μM Myelin basic protein (MBP). The following components were added in a white polystyrene Costar plate up to a final volume of 30 μL: 10 μL inhibitor dissolved in DMSO/water 50/50, and 20 μL GmhA of E. coli in AB. After 30 min of pre-incubation at room temperature, 30 μL of Substrates mix in AB were added in each well to a final volume of 60 μL. This reaction mixture was then composed of 2 nM GmhA, 3 μM sedoheptulose-7-phosphate (Sigma), 3 μM ATP (Sigma) and 50 nM HldE of E. coli in assay buffer. After 30 min of incubation at room temperature, 100 μL of the revelation mix were added to a final volume of 160 μL, including the following constituents at the respective final concentrations: 10000 light units/ml luciferase (Sigma), 20 μM D-luciferin (Sigma), 100 μM N-acetylcysteamine (Aldrich). Luminescence intensity was immediately measured on Luminoskan (Thermofischer) and converted into inhibition percentages. For IC50 determinations, the inhibitor was tested at 6 to 10 different concentrations, and the related inhibitions were fitted to a classical langmuir equilibrium model using XLFIT (IDBS).

Inhibition of the Enzymatic Activity of HldE-K (Luminescent Assay On Kinase Activity):

The assay buffer “AB” contained 50 mM Hepes pH7.5, 1 mM MnCl₂, 25 mM KCl, 0.012% Triton-X100 and 1 mM dithiothreitol (DTT) and 0.1 μM Myelin basic protein (MBP). The following components were added in a white polystyrene Costar plate up to a final volume of 30 μL: 10 μL inhibitor dissolved in DMSO/water 50/50, and 20 μL HldE of E. coli in AB. After 30 min of pre-incubation at room temperature, 30 μL of Substrates mix in AB were added in each well to a final volume of 60 μL. This reaction mixture was then composed of 3 nM HldE, 0.2 μM β-heptose-7-phosphate (custom synthesis) and 0.2 μM ATP (Sigma) in assay buffer. After 30 min of incubation at room temperature, 200 μL of the revelation mix were added to a final volume of 2604, including the following constituents at the respective final concentrations: 5000 light units/ml luciferase (Sigma), 30 μM D-luciferin (Sigma), 100 μM N-acetylcysteamine (Aldrich). Luminescence intensity was immediately measured on Luminoskan (Thermofischer) and converted into inhibition percentages. For IC50 determinations, the inhibitor was tested at 6 to 10 different concentrations, and the related inhibitions were fitted to a classical Langmuir equilibrium model using XLFIT (IDBS).

Inhibition of E. Coli C7 (018:K1:H7) LPS Biosynthesis:

Principle: E. Coli C7 (018:K1:H7) is a Newborm Meningitidis E. coli (NMEC) strain which displays a typical LPS made of Lipid A successively branched with the inner and outer core oligosaccharides, and finally with the O-antigen repeats. The inner core contains several heptose residues. An inhibitor of the LPS heptosylation pathway should therefore reduce dramatically the size of LPS from full-length to the so-called ‘Re-LPS’ limited to lipid A branched with 2 Kdo residues. A simple way of monitoring LPS size and composition consists in running LPS gel electrophoresis (FIG. 1): a wild type E. coli strain displays several bands including those for full and core LPS but none for Re-LPS. On the contrary, a delta-hldE mutant defective for LPS-heptosylation biosynthesis displays only the Re-LPS band.

Bacterial culture: The effect of heptosylation inhibitors on E. coli LPS was assessed as described below. The compounds to be tested were prepared in deionised water/DMSO (50/50) solutions and added (25 μL) in sterile culture microtubes. The strain used in this study was E. coli C7 (018:K1:H7). The bacteria were isolated on tryptic soy agar (TSA) over-night. Isolated colonies were cultured in 10 ml of Luria-Bertani medium (LB) at 37° C. up to an optical density of typically 0.15. These exponentially growing bacteria were finally diluted to 5e5 cfu/ml and added in each well (225 μL) for incubation with the compounds at 37° C. for approximately 5 hours, up to an optical density of ≈0.2-0.4. Some test compounds e.g. phospho-sugars required Glucose-6-Phosphate (G6P, from Sigma) to be added in the culture medium in order to activate their active transport into the bacterial cytosol via the phospho-sugar transporter UhpT. This was achieved by adding in the culture tube 2.5 μL of a 10 mM water solution of G6P (100 μM final concentration).

LPS extraction: Bacterial cultures were normalized via OD determination, pelleted and washed with 1 ml Phosphate-Buffer-Saline (PBS). The pellets were then denatured for 10 min at 95-100° C. in 50 μl of Sodium-Dodecyl-Sulfate 0.2% (SDS), beta-mercaptoethanol 1%, Glycerol 36%, Tris pH7.4 30 mM and bromophenol blue 0.001%. Samples were cooled down to room temperature, supplemented with 1.5 μl of proteinase K at 20 mg/ml, incubated for 1H at 55° C. and centrifuged for 30 min at 13000 rpm at 25° C. The resulting supernatant, containing LPS was finally analysed by SDS-PAGE electrophoresis.

LPS SDS-PAGE electrophoresis: Polyacrylamide gels (16%/4% acrylamide for separation and concentration respectively) were prepared, loaded with 8 μl of LPS extracts and migrated.

Silver staining: Gels were incubated overnight in 5% acetic acid/40% ethanol/deionised water, treated by 1% periodic acid/5% acetic acid for 15 min, washed 4 times for 10 min in deionised water and finally incubated for 18 min in the dark in a silver nitrate solution composed of 56 ml NaOH 0.1N, 4 ml ammoniac 33%, 45 ml AgNO3 5% (Tsai and Frasch) and 195 ml deionised water. Gels were then washed extensively in deionised water for 30 min and incubated for 10-15 min (up to LPS bands apparition) in the revelation mix composed of 300 ml deionised water, 300 μl formaldehyde 36.5% (Fluka) and 100 μl citric acid 2.3M. The revelation was stopped by incubating the gels in acetic acid 10% for 5 min. Gels were finally washed in deionised water, numerized with a Samsung PL51 camera and analysed by ImageJ software. The percentage of inhibition of LPS heptosylation was defined as the relative area of the Re-LPS band compared to the cumulated areas of Re-LPS and Core-LPS bands.

Inhibitory Activities of Selected Compounds:

Compounds described in examples 1, 2, 3, 6, 7, 8, 11, 12 and 13 display IC50 values <100 μM on GmhA. Compounds described in examples 1, 2, 6, 7, 8, 11, 12 and 13 display IC50 values <100 μM on HldE-K. Compounds described in examples 6, 7 and 13 display in the presence of 100 μM G6P at least 30% inhibition of E. coli C7 LPS heptosylation at concentrations <10 mM. Compound described in example 10 displays without G6P at least 30% inhibition of E. coli C7 LPS heptosylation at concentrations <10 mM.

1. Compounds having the general formula (I) 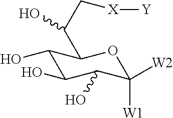
wherein,
Carbon-2 is in D-manno-heptose or D-gluco-heptose configuration or as a mixture of both;
Carbon-6 is in D-glycero-heptose configuration;
X is O, S, CH₂, CHF, CF₂ or NH;
Y is H or P(O)(OZ1)(OZ2), P(O)(OZ1)(NHZ2) or SO₂(OZ1);
Z1 and Z2, identical or different, are H, (C₁-C₆)alkyl, n-octadecanoyl, (C₁-C₆)fluoroalkyl, CH₂O(CO)O(C₁-C₆)alkyl, CH((C₁-C₆)alkyl)O(CO)O(C₁-C₆)alkyl, CH₂O(CO)O(C₁-C₆)fluoroalkyl, CH₂O(CO)(C₁-C₆)alkyl, CH((C₁-C₆)alkyl)O(CO)(C₁-C₆)alkyl, CH₂O(CO)(C₁-C₆)fluoroalkyl, CH₂CH(O-n-decanoyl)CH₂S-n-dodecanoyl, (C₂-C₆) alkenyl, (C₂-C₆) alkynyl, CH₂CH₂S(CO)(C₁-C₆)alkyl, CH((C₁-C₆)alkyl)(CO)O(C₁-C₆)alkyl, CH₂(CO)O(C₁-C₆)alkyl; phenyl optionally substituted by one or several identical or different groups R; 4-6 membered monocyclic saturated or unsaturated heterocycle containing 1-3 heteroatoms selected from N, O and S, optionally substituted by one or several identical or different groups R; or mono-, di- or trivalent cation such as lithium, sodium, potassium, magnesium, calcium, cesium, barium, ammonium, to form a phosphate salt; Z1 and Z2 may form a 4-10 membered cycle with each other, optionally including those selected from the group comprising CH₂CH₂CH(m-chlorophenyl or pyridyl), CH₂CH₂CH(O(CO)(C₁-C₆)alkyl);
W1 and W2 identical or different, optionally linked with each other, are selected from the group consisting of H, F, CN, (C₁-C₆)alkyl, (C₁-C₆)alkyl-ORₐ, (C₁-C₆)alkyl-O(C₁-C₆)alkyl, (C₁-C₆)fluoroalkyl, (C₂-C₆)alkenyl, (C₂-C₆)alkynyl, phenyl, 4-10 membered monocyclic or bicyclic saturated or unsaturated heterocycle containing 1-5 heteroatoms selected from N, O and S; CO₂Rₐ, CORₐ, CONRₐR_{b}, OCORₐ, ORₐ, NRₐR_{b}, CRₐ═NOR_{b}, NRₐCOR_{b}, NRₐCOOR_{b}, OCONRₐR_{b}, OCO₂Rₐ, NRₐCONR_{b}R_{c}, NRₐSO₂R_{b}, S(O)ₙRₐ, and SO₂NRₐR_{b}, CONRₐOR_{b}, N(OR_{b})CORₐ all the above members of the group representing W1 or W2 being optionally substituted by one, two or three identical or different groups R, which may form a cycle with each other;
Rₐ, R_{b} and Rₑ, identical or different, are selected from the group consisting of H, (C₁-C₆)alkyl, C₁-C₆)fluoroalkyl, (C₂-C₆)alkenyl, (C₂-C₆)alkynyl, phenyl, benzyl and 4-6 membered monocyclic saturated or unsaturated heterocycle containing 1-3 heteroatoms selected from N, O and S; Rₐ, R_{b} and Rₑ may form a cycle with each other optionally including 1-3 heteroatoms selected from N, O and S, illustrative examples of saturated nitrogen containing heterocycles within the definition of NRaRb include those selected from the group comprising, pyrrolidinyl, oxazolidinyl, thiazolidinyl, piperidinyl, piperazinyl and morpholinyl.
R is selected from the group consisting of halogen, CN, (C₁-C₆)alkyl, (C₁-C₆)fluoroalkyl, (C₂-C₆)alkenyl, (C₂-C₆)alkynyl, phenyl, 4-10 membered monocyclic or bicyclic saturated or unsaturated heterocycle containing 1-5 heteroatoms selected from N, O and S; CO₂Rₐ, CORₐ, CONRₐR_{b}, OCORₐ, ORₐ, NRₐR_{b}, CRₐ═NOR_{b}, NRₐCOR_{b}, NRₐCOOR_{b}, OCONRₐR_{b}, OCO₂Rₐ, NRₐCONR_{b}R_{C}, NRₐSO₂R_{b}, S(O)ₙRₐ, and SO₂NRₐR_{b}, CONRₐOR_{b}, N(OR_{b})CORₐ; all the above members of the group representing R being optionally substituted by one or several identical or different groups R′, which may form a cycle with each other;
R′ is selected from the group consisting of halogen, CN, (C₁-C₆)alkyl, (C₁-C₆)fluoroalkyl, (C₂-C₆)alkenyl, (C₂-C₆)alkynyl, phenyl, 4-10 membered monocyclic or bicyclic saturated or unsaturated heterocycle containing 1-5 heteroatoms selected from N, O and S; CO₂Rₐ, CORₐ, CONRₐR_{b}, OCORₐ, ORₐ, NRₐR_{b}, CRₐ═NOR_{b}, NRₐCOR_{b}, NRₐCOOR_{b}, OCONRₐR_{b}, OCO₂Rₐ, NRₐCONR_{b}R_{c}, NRₐSO₂R_{b}, S(O)ₙRₐ, and SO₂NRₐR_{b}, CONRₐOR_{b}, N(OR_{b})CORₐ;
n is 0, 1 or 2;
their N-oxide derivatives,
in their racemic, scalemic (non racemic mixtures), enantiomeric or geometric forms, and their addition salts thereof with acids and bases,
to the exclusion of the following compounds:
Methyl (6R/S)—C-ethyl-α-D-glycero-pyranoside;
D/L-Glycero-D-manno-heptose-7-phosphate, and methyl α-D-manno-heptopyranoside-7-phosphate;
—O-L-glycero-α-D-manno-heptopyranosyl-(1→7)-L-glycero-D-manno-heptopyranose Methyl 7-O-L-glycero-α-D-manno-heptopyranosyl-L-glycero-α-D-matmo-heptopyranose;
Methyl 7-O-L-glycero-α-D-manno-heptopyranosyl-L-gdycero-α-D-manno-heptopyranoside;
Allyl 6-O-(L-glycero-α-D-manno-heptopyranosyl)-α-D-glycero-pyrano side;
Methyl 7-O-(2-aminoethyl)phosphoryl-L-glycero-α-D-manno-heptopyranoside; and
with the proviso that when X is O and Y is H, W1 and W2 may not form a double bond with each other when W1 is a linking bond and W2 is (O)(D/L-Glycero-D-manno-hepto-1,5-pyranone); when W1 is H then W2 is different from OH, OCH₃, CH₂CH(CH₃)OH, CH₂C(O)CH3, or when W2 is H then W1 is different from OH, OCH₃, OBn, OCH₂CH═CH₂, CH₂CH(CH₃)OH, CH₂C(O)CH₃, SC₂H₅, (N-Benzylcarbamoyl)-3-propyloxy, 3-(Perfluorooctyppropanyl-oxybutanyloxy.

2. The compounds according to claim 1, in which at least one of W1 and W2 is H, and X is O, S, CH₂ or NH, and Y is H, P(O)(OZ1)(OZ2) or P(O)(OZ1)(NHZ2).

3. The compounds according to claim 1, in which X is O and Y is H.

4. The compounds according to claim 1, in which W1 and W2 are H.

5. The compounds according to claim 1, in which X is CH₂, CHF or CF₂ and Y is P(O) (OZ1) (OZ2).

6. The compounds according to claim 1, which are drugs.

7. The compounds according to claim 1, which are inhibitors of bacterial heptose synthesis.

8. Pharmaceutical compositions comprising a therapeutically effective amount of at least one compound of formula (I) in association with a pharmaceutically acceptable carrier, the formula (I) comprising: 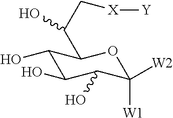
wherein
Carbon-2 is in D-manno-heptose or D-glycero-heptose configuration or as a mixture of both;
Carbon-6 is in D-glycero-heptose configuration.,
X is O, S, CH₂, CHF, CF, or NH;
Y is H or P(O)(OZ1)(OZ2), P(O)(OZ1)(NHZ2) or SO₂(OZ1);
Z1 and Z2, identical or different, are H, (C₁-C₆)alkyl, n-octadecanoyl, (C₁-C₆)fluoroalkyl, CH₂O(CO)O(C₁-C₆)alkyl, CH((C₁-C₆)alkyl)O(CO)O(C₁-C₆)alkyl, CH₂O(CO)O(C₁-C₆)fluoroalkyl, CH₂O(CO)(C₁-C₆)alkyl, CH((C₁-C₆)alkyl)O(CO)(C₁-C₆)alkyl, CH₂O(CO)(C₁-C₆)fluoroalkyl, CH₂CH(O-n-decanoyl)CH₂S-n-dodecanoyl, (C₂-C₆) alkenyl, (C₂-C₆)alkynyl, CH₂CH₂S(CO)(C₁-C₆)alkyl, CH((C₁-C₆)alkyl)(CO)O(C₁-C₆)alkyl, CH₂(CO)O(C₁-C₆)alkyl; phenyl; 4-6 membered monocyclic saturated or unsaturated heterocycle containing 1-3 heteroatoms selected from N, O and S;
W1 and W2 identical or different, are selected from the group consisting of H, F, CN, (C₁-C₆)alkyl, (C₁-C₆)alkyl-ORₐ, (C₁-C₆)alkyl-O(C₁-C₆)alkyl, (C₁-C₆)fluoroalkyl, (C₂-C₆)alkenyl, (C₂-C₆)alkynyl, phenyl, 4-10 membered monocyclic or bicyclic saturated or unsaturated heterocycle containing 1-5 heteroatoms selected from N, O and S; CO₂Rₐ, CORₐ, CONRₐR_{b}, OCORₐ, ORₐ, NRₐR_{b}, CRₐ═NOR_{b}, NRₐCOR_{b}, NRₐCOOR_{b}, OCONRₐR_{b}, OCO₂Rₐ, NRₐCONR_{b}R_{c}, NRₐSO₂R_{b}, S(O)ₙRₐ, and SO₂NRₐR_{b}, CONRₐOR_{b}, N(OR_{b})CORₐ all the above members of the group representing W1 or W2;
Rₐ, R_{b} and R_{c}, identical or different, are selected from the group consisting of H, (C₁-C₆)alkyl, C₁-C₆)fluoroalkyl, (C₂-C₆)alkenyl, (C₂-C₆)alkynyl, phenyl, benzyl and 4-6 membered monocyclic saturated or unsaturated heterocycle containing 1-3 heteroatoms selected from N, O and S; Rₐ, R_{b} and R_{c} may form a cycle with each other.
R is selected from the group consisting of halogen, CN, (C₁-C₆)alkyl, (C₁-C₆)fluoroalkyl, (C₂-C₆)alkenyl, (C₂-C₆)alkynyl, phenyl, 4-10 membered monocyclic or bicyclic saturated or unsaturated heterocycle containing 1-5 heteroatoms selected from N, O and S; CO₂Rₐ, CORₐ, CONRₐR_{b} OCORₐ, ORₐ, NRₐR_{b}, CRₐ═NOR_{b}, NRₐCOR_{b}, NRₐCOOR_{b}, OCONRₐR_{b}, OCO₂Rₐ, NRₐCONR_{b}R_{c}, NRₐSO₂R_{b}, S(O)ₙRₐ and SO₂NRₐR_{b}, CONRₐOR_{b}, N(OR_{b})CORₐ; all the above members of the group representing R;
R′ is selected from the group consisting of halogen, CN, (C₁-C₆)alkyl, (C₁-C₆)fluoroalkyl, (C₂-C₆)alkenyl, (C₂-C₆)alkynyl, phenyl, 4-10 membered monocyclic or bicyclic saturated or unsaturated heterocycle containing 1-5 heteroatoms selected from N, O and S; CO₂Rₐ, CORₐ, CONRₐR_{b} OCORₐ, ORₐ, NRₐR_{b}, CRₐ═NOR_{b}, NRₐCOR_{b}, NRₐCOOR_{b}, OCONRₐR_{b}, OCO₂Rₐ, NRₐCONR_{b}R_{c}, NRₐSO₂R_{b}, S(O)ₙRₐ and SO₂NRₐR_{b}, CONRₐOR_{b}, N(OR_{b})CORₐ;
n is 0, 1 or 2;
their N-oxide derivatives,
in their racemic, scalemic (non racemic mixtures), enantiomeric or geometric forms,
and their addition salts thereof with acids and bases,
to the exclusion of the following compounds:
Methyl (6R/S)—C-ethyl-α-D-glycero-pyranoside;
D/L-Glycero-D-manno-heptose-7-phosphate, and methyl α-D-manno-heptopyranoside-7 phosphate;
—O-L-glycero-α-D-manno-heptopyranosyl-(1→7)-L-glycero-D-manno-heptopyranose Methyl 7-O-L-glycero-α-D-manno-heptopyranosyl-L-glycero-α-D-manno-heptopyranose;
Methyl 7-O-L-glycero-α-D-manno-heptopyranosyl-L-glycero-α-D-manno-heptopyranoside;
Allyl 6-O-(L-glycero-α-D-manno-heptopyranosyl)-α-D-gluco-pyranoside;
Methyl 7-O-(2-aminoethyl)phosphoryl-L-glycero-α-D-manno-heptopyranoside; and
with the proviso that when X is O and Y is H, W1 and W2 may not form a double bond with each other when W1 is a linking bond and W2 is (O)(D/L-Glycero-D-manno-hepto-1,5-pyranone); when W1 is H then W2 is different from OH, OCH₃ CH₇CH(CH₂)OH, CH₂C(O)CH3, or when W2 is H then W1 is different from OH, OCH₃, OBn, OCH₂CH═CH₂; and CH₂CH(CH₃)OH, CH₂C(O)CH₃, SC₂H₅, (N-Benzylcarbamoyl)-3-propyloxy, 3-(Perfluorooctyl)propanyl-oxybutanyloxy.

9. The pharmaceutical compositions according to claim 8 formulated to be administered under oral, parenteral, and injectable routes, with individual doses appropriate for the patient to be treated.

10. The pharmaceutical compositions according to claim 8, in combination with at least one antibacterial.

11. The pharmaceutical compositions according to claim 8, in combination with at least one antivirulence agent.

12. The pharmaceutical compositions according to claim 8, in combination with one or more drug(s) reinforcing the host innate immunity.

13. The pharmaceutical compositions according to claim 8, preventing or therapeutically treating severe infections due to Gram-negative bacteria able to disseminate in blood such as the non-limiting following species (spp.): Escherichia coli, Enterobacter, Salmonella, Shigella, Pseudomonas, Acinetobacter, Neisseria, Klebsiella, Serratia, Citrobacter, Proteus, Yersinia, Haemophilus, Legionella, Moraxella and Helicobacter pylori.

14. A method of preventing or treating a bacterial infection in a patient in need thereof comprising administering to the patient a pharmaceutical composition comprising a compound of general formula (I) 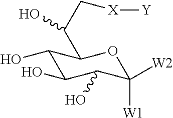
wherein,
Carbon-2 is in D-manno-heptose or D-gluco-heptose configuration or as a mixture of both;
Carbon-6 is in D-glycero-heptose configuration;
Y is H or P(O)(OZ1)(OZ2), P(O)(OZ1)(NHZ2) or SO₂(OZ1);
Z1 and Z2, identical or different, are H, (C₁-C₆)alkyl, n-octadecanoyl, (C₁-C₆)fluoroalkyl, CH₂O(CO)O(C₁-C₆)alkyl, CH((C₁-C₆)alkyl)O(CO)O(C₁-C₆)alkyl, CH₂O(CO)O(C₁-C₆)fluoroalkyl, CH₂O(CO)(C₁-C₆)alkyl, CH((C₁-C₆)alkyl)O(CO)(C₁-C₆)alkyl, CH₂O(CO)(C₁-C₆)fluoroalkyl, CH₂CH(O-n-decanoyl)CH₂S-n-dodecanoyl, (C₂-C₆) alkenyl, (C₂-C₆) alkynyl, CH₂CH₂S(CO)(C₁-C₆)alkyl, CH((C₁-C₆)alkyl)(CO)O(C₁-C₆)alkyl, CH₂(CO)O(C₁-C₆)alkyl; 4-6 membered monocyclic saturated or unsaturated heterocycle containing 1-3 heteroatoms selected from N, O and S;
W1 and W2, identical or different, are selected from the group consisting of H, F, CN, (C₁-C₆)alkyl, (C₁-C₆)alkyl-ORₐ, (C₁-C₆)alkyl-O(C₁-C₆)alkyl, (C₁-C₆)fluoroalkyl, (C₂-C₆)alkenyl, (C₂-C₆)alkynyl, phenyl, 4-10 membered monocyclic or bicyclic saturated or unsaturated heterocycle containing 1-5 heteroatoms selected from N, O and S; CO₂Rₐ, CORₐ, CONRₐR_{b}, OCORₐ, ORₐ, NRₐR_{b}, CRₐ═NOR_{b}, NRₐCOR_{b}, NRₐCOOR_{b}, OCONRₐR_{b}, OCO₂Rₐ, NRₐCONR_{b}R_{c}, NRₐSO₂R_{b}, S(O)ₙRₐ, and SO₂NRₐR_{b}, CONRₐOR_{b}, N(OR_{b})CORₐ all the above members of the group representing W1 or W2;
Rₐ, R_{b} and R_{c}, identical or different, are selected from the group consisting of H, (C₁-C₆)alkyl, C₁-C₆)fluoroalkyl, (C₂-C₆)alkenyl, (C₂-C₆)alkynyl, phenyl, benzyl and 4-6 membered monocyclic saturated or unsaturated heterocycle containing 1-3 heteroatoms selected from N, O and S; Rₐ, R_{b} and R_{c} may form a cycle with each other
R is selected from the group consisting of halogen, CN, (C₁-C₆)alkyl, (C₁-C₆)fluoroalkyl, (C₂-C₆)alkenyl, (C₂-C₆)alkynyl, phenyl, 4-10 membered monocyclic or bicyclic saturated or unsaturated heterocycle containing 1-5 heteroatoms selected from N, O and S; CO₂Rₐ, CORₐ, CONRₐR_{b}, OCORₐ, ORₐ, NRₐR_{b}, CRₐ═NOR_{b}, NRₐCOR_{b}, NRₐCOOR_{b}, OCONRₐR_{b}, OCO₂Rₐ, NRₐCONR_{b}R_{c}, NRₐSO₂R_{b}, S(O)ₙRₐ, and SO₂NRₐR_{b}, CONRₐOR_{b}, N(OR_{b})CORₐ; all the above members of the group representing R;
R′ is selected from the group consisting of halogen, CN, (C₁-C₆)alkyl, (C₁-C₆)fluoroalkyl, (C₂-C₆)alkenyl, (C₂-C₆)alkynyl, phenyl, 4-10 membered monocyclic or bicyclic saturated or unsaturated heterocycle containing 1-5 heteroatoms selected from N, O and S; CO₂Rₐ, CORₐ, CONRₐR_{b}, OCORₐ, ORₐ, NRₐR_{b}, CRₐ═NOR_{b}, NRₐCOR_{b}, NRₐCOOR_{b}, OCONRₐR_{b}, OCO₂Rₐ, NRₐCONR_{b}R_{c}, NRₐSO₂R_{b}, S(O)ₙRₐ, and SO₂NRₐR_{b}, CONRₐOR_{b}, N(OR_{b})CORₐ;
n is 0, 1 or 2;
their N-oxide derivatives,
the compound in their racemic, scalemic (non racemic mixtures), enantiomeric or geometric forms,
and their addition salts thereof with acids and bases,
to the exclusion of the following compounds:
Methyl (6R/S)—C-ethyl-α-D-gluco-pyranoside;
D/L-Glycero-D-manno-heptose-7-phosphate, and methyl α-D-manno-heptopyranoside-7 phosphate;
—O-L-glycero-α-D-manno-heptopyranosyl-(1→7)-L-glycero-D-manno-heptopyranose Methyl 7-O-L-glycero-α-D-manno-heptopyranosyl-L-glycero-α-D-manno-heptopyranose;
Methyl 7-O-L-glycero-α-D-manno-heptopyranosyl-L-glycero-α-D-manno heptopyranoside;
Allyl 6-O-(L-glycero-α-D-manno-heptopyranosyl)-α-D-gluco-pyranoside;
Methyl 7-O-(2-aminoethyl)phosphoryl-L-glycero-α-D-manno-heptopyranoside; and
with the proviso that when X is O and Y is H, W1 and W2 may not form a double bond with each other when W1 is a linking bond and W2 is (O)(D/L-Glycero-D-manno-hepto-1,5-pyranone); when W1 is H then W2 is different from OH, OCH₃, CH₂CH(CH₃)OH, CH₂C(O)CH3, or when W2 is H then W1 is different from OH, OCH₃, OBn, OCH₂CH═CH₂; and CH₂CH(CH₃)OH, CH₂C(O)CH₃, SC₂H₅, (N-Benzylcarbamoyl)-3-propyloxy, 3-(Perfluorooctyl)propanyl-oxybutanyloxy.

15. The method according to claim 14, wherein W1 and/or W2 is H, and X is O, S, CH₂ or NH, and Y is H, P(O)(OZ1)(OZ2) or P(O)(OZ1)(NHZ2).

16. The method according to claim 14, wherein X is O and Y is H.

17. The method according to claim 14, wherein W1 and W2 are H.

18. The method according to claim 14, wherein X is CH₂, CHF or CF₂ and Y is P(O) (OZ1) (OZ2).

19. The method according to claim 14, wherein Rₐ, R_{b} and/or R is a saturated nitrogen containing heterocycle of formula NRaRb selected from the group consisting of pyrrolidinyl, oxazolidinyl, thiazolidinyl, piperidinyl, piperazinyl, and morpholinyl.

20. The method according to claim 14, wherein the compound is an inhibitor of bacterial heptose synthesis.

21. The method according to claim 14, wherein the pharmaceutical composition further comprises a pharmaceutically acceptable carrier.

22. The method according to claim 14, further comprising administering the pharmaceutical compound orally, parenterally, or by injection at a therapeutically effective dose.

23. The method according to claim 14, further comprising administering at least one additional antimicrobial compound.

24. The method according to claim 23, wherein the antimicrobial is a natural, hemisynthetic or synthetic antibacterial or antivirulence agent.

25. The method according to claim 24, wherein the antimicrobial is a peptide.

26. The method according to claim 14, further comprising administering one or more drugs for reinforcing the patient's innate immunity.

27. The method according to claim 14, wherein the bacterial infection comprises gram negative bacteria.

28. The method according to claim 27, wherein the gram negative bacteria are Escherichia coli, Enterobacter, Salmonella, Shigella, Pseudomonas, Acinetobacter, Neisseria, Klebsiella, Serratia, Citrobacter, Proteus, Yersinia, Haemophilus, Legionella, Moraxella, Helicobacter pylori or combinations thereof.

29. The method according to claim 14, wherein the patient is a human.

30. The method according to claim 14, further comprising administering 0.1 to 10 g of the compound per day.
